(19) United States
(12) Patent Application Publication
(10) Pub. No.: US 2007/0178475 A1
(43) Pub. Date: Aug. 2, 2007

(54) Novel human polynucleotides and polypeptides encoded thereby

(76) Inventors: Michael C. Nehls, Stockdorf (DE); Brian Zambrowicz, The Woodlands, TX (US); Arthur T. Sands, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 11/128,224

(22) Filed: May 12, 2005

Related U.S. Application Data
(63) Continuation-in-part of application No. 11/026,159, filed on Dec. 30, 2004, now abandoned.
Application No. 11/026,159 is a continuation of application No. 09/398,253, filed on Sep. 17, 1999, now abandoned.
(60) Provisional application No. 60/100,917, filed on Sep. 17, 1998.

(51) Int. Cl. C12Q 1/68; G06F 19/00; C07H 21/04

(52) U.S. Cl. 435/6; 536/23.1; 702/20

(57) ABSTRACT

Novel human polynucleotides are disclosed that correspond to human gene trapped sequences, or GTSs. The disclosed GTSs are useful for gene discovery and as markers for, inter alia, gene expression analysis, identifying and mapping the coding regions of the mammalian, and particularly human, genome, forensic analysis, and determining the genetic basis of human disease.

### 1.0 CROSS-REFERENCE TO RELATED APPLICATIONS

The present application is a continuation-in-part of: co-pending U.S. application Ser. No. 11/026,159, filed on Dec. 30, 2004, which is a continuation of U.S. application Ser. No. 09/398,253, filed on Sep. 17, 1999, abandoned, which claims the benefit of U.S. Provisional Application Ser. No. 60/100,917, which was filed on Sep. 17, 1998; co-pending U.S. application Ser. No. 10/914,016, filed on Aug. 5, 2004, which is a continuation of U.S. application Ser. No. 09/417,522, filed on Oct. 13, 1999, abandoned, which claims the benefit of U.S. Provisional Application Ser. No. 60/104,292, which was filed on Oct. 14, 1998; co-pending U.S. application Ser. No. 10/911,704, filed on Aug. 4, 2004, which is a continuation of U.S. application Ser. No. 09/421,813, filed on Oct. 20, 1999, abandoned, which claims the benefit of U.S. Provisional Application Ser. No. 60/104,977, which was filed on Oct. 20, 1998; co-pending U.S. application Ser. No. 10/914,037, filed on Aug. 6, 2004, which is a continuation of U.S. application Ser. No. 09/428,674, filed on Oct. 27, 1999, abandoned, which claims the benefit of U.S. Provisional Application Ser. No. 60/106,442, which was filed on Oct. 30, 1998; co-pending U.S. application Ser. No. 09/560,863, filed on Apr. 28, 2000, which claims the benefit of U.S. Provisional Application Ser. No. 60/132,408, which was filed on Apr. 30, 1999; and co-pending U.S. application Ser. No. 09/563,817, filed on May 3, 2000, which claims the benefit of U.S. Provisional Application Ser. No. 60/132,343, which was filed on May 4, 1999; each of which are herein incorporated by reference in their entirety.

### 2.0 CROSS-REFERENCE TO SEQUENCE LISTING SUBMITTED ON COMPACT DISC

The present application contains a Sequence Listing of SEQ ID NOS:1-5,504, in file “seqlist.txt” (3,137,536 bytes), created on May 12, 2005, submitted herewith on duplicate compact disc (Copy 1 and Copy 2), which is herein incorporated by reference in its entirety.

### 3.0 BACKGROUND OF THE INVENTION

The Human Genome Project and privately financed ventures have succeeded in sequencing the human genome. The hope was that a comprehensive representation of the human genome would thus be available for biomedical analysis. However, the data resulting from these efforts largely comprise human genomic sequence, of which only a fraction encodes expressed sequence information. Although sophisticated computer-assisted exon identification programs can be applied to such genomic sequence data, the computer predictions require verification by laboratory analysis to actually identify the coding regions of the genome, and to identify exon splice junctions. Thus, the availability of cDNA information will contribute notably to the value of human genomic sequence, since cDNA sequence provides a direct indication of the presence of transcribed sequences, as well as the location of splice junctions.

Thus, sequencing of cDNA libraries to obtain expressed sequence tags (or ESTs) that identify exons expressed within a given tissue, cell, or cell line, is currently in progress. As a consequence of these efforts, a large number of EST sequences are presently compiled in public and privately held databases. However, the present EST paradigm is inherently limited by the levels and extent of mRNA production within a given cell. A related problem is the lack of cDNA sources from specific tissue and developmental expression profiles. In addition, some genes are typically only active under certain physiological conditions, or are generally expressed at levels below or near the threshold necessary for cDNA cloning and detection, and are therefore not effectively represented in current cDNA libraries.

Researchers have partially addressed these issues by using phage vectors to clone genomic sequences such that internal exons are trapped (Nehls et al., Current Biology 4:983-989, 1994, and Nehls et al., Oncogene 9:2169-2175, 1994). However, such libraries require the random cloning of genomic DNA into a suitable cloning vector in vitro, followed by reintroduction of the cloned DNA in vivo in order to express and splice the cloned genes prior to producing the cDNA library. Additionally, such methods can only “trap” the internal exons of genes. Consequently, genes containing a single exon or a single intron are typically not trapped by traditional methods of exon trapping.

### 4.0 SUMMARY OF THE INVENTION

The present application discloses novel nucleic acid sequences that partially define the scope of human exons that can be trapped and identified by the disclosed vectors and methods, and are useful, inter alia, for identifying the organization of coding regions of the human genome. The Sequence Listing is a compilation of nucleotide sequences obtained by sequencing a human gene trap library that at least partially identifies the genes in the target cell genome that can be trapped by the described gene trap vectors (i.e., the repertoire of genes that are active, or not inactivated).

The invention thus provides isolated and purified novel human cDNAs produced using gene trap technology. The novel human gene trapped sequences (GTSs) of the invention are disclosed as SEQ ID NOS:10-5,504 in the Sequence Listing, submitted herewith on compact disc. The invention further contemplates the use of one or more of the instant GTSs, or portions thereof, to isolate cDNAs, genomic clones, or full-length genes/polynucleotides, or homologs, heterologs, paralogs, or orthologs thereof, that are capable of hybridizing to one or more of the disclosed GTSs, or their complementary sequences, under stringent conditions.

The present invention also contemplates methods of analyzing biopolymer (e.g., oligonucleotides, polynucleotides, oligopeptides, peptides, polypeptides, proteins, etc.) sequence information, comprising the steps of loading a first biopolymer sequence into or onto an electronic data storage medium (e.g., digital or analogue versions of electronic, magnetic, or optical memory, and the like), and comparing the first biopolymer sequence to at least a portion of one of the polynucleotide sequences, or amino acid sequences encoded thereby, that is first disclosed in, or otherwise unique to, SEQ ID NOS:10-5,504. Typically, the instant polynucleotide sequences, or amino acid sequences encoded thereby, will also be present on, or loaded into or onto a form of electronic data storage medium, or transferred therefrom, concurrent with or prior to comparison with the first biopolymer sequence.

Another embodiment of the invention is the use of an oligonucleotide or polynucleotide sequence first disclosed in at least a portion of at least one of the GTS sequences of SEQ ID NOS:10-5,504 as a hybridization probe or for chromosome mapping. Of particular interest is the use of such sequences in conjunction with a solid support matrix/substrate (resins, beads, membranes, plastics, polymers, metal or metallized substrates, crystalline or polycrystalline substrates, etc.). Of particular note are spatially addressable arrays (gene chips, microtiter plates, etc.) of polynucleotides, wherein at least one of the polynucleotides on the spatially addressable array comprises an oligonucleotide or polynucleotide sequence first disclosed in at least one of SEQ ID NOS:10-5,504. Similarly, one or more oligonucleotide probes based on, or otherwise incorporating, sequences first disclosed in any one of SEQ ID NOS:10-5,504, can be used to obtain novel gene sequence via the polymerase chain reaction (PCR) or cycle sequencing. Similar oligonucleotide hybridization probes can also comprise sequence that is complementary to a portion of a sequence that is first disclosed in, or preferably unique to, at least one of the GTS polynucleotides disclosed in the Sequence Listing. The oligonucleotide probes will generally comprise between about 8 nucleotides and about 80 nucleotides, preferably between about 15 and about 40 nucleotides, and more preferably between about 20 and about 35 nucleotides.

Moreover, an oligonucleotide or polynucleotide sequence first disclosed in at least one of the GTS sequences of SEQ ID NOS:10-5,504 can be incorporated into a phage display system that can be used to screen for proteins, or other ligands, that are capable of binding an amino acid sequence encoded by an oligonucleotide or polynucleotide sequence first disclosed in at least one of the GTS sequences of SEQ ID NOS:10-5,504.

An additional embodiment of the present invention is a library comprising individually isolated linear DNA molecules corresponding to at least a portion of the described human GTSs, which are useful for synthesizing physically contiguous sequences of overlapping GTSs by, for example, PCR. The invention also provides for antisense molecules that comprise at least a portion of a sequence that is first disclosed in, or unique to, at least one of the GTS polynucleotides.

The present invention further contemplates purified polypeptides in which at least a portion of a polypeptide is encoded by, or first disclosed in, at least a portion of a GTS of the present invention. The invention also relates to naturally occurring polynucleotides comprising the disclosed GTSs that are expressed by promoter elements other than the promoter elements that normally express the GTSs in human cells (i.e., gene activated GTSs). Such promoter elements can be directly incorporated into the cellular genome, or recombinantly engineered upstream (for example, about 50, about 75, or at least 100 to 130 bases upstream) from at least a portion of a GTS of the present invention, or a complement thereof. Additional embodiments of the present invention are recombinantly engineered expression vectors that comprise at least a portion of the disclosed GTSs, or complements thereof.

The unique sequences described in SEQ ID NOS:10-5,504 are also useful for the identification of coding sequence, the mapping of a unique gene to a particular chromosome, and can also be used in addressable arrays, such as gene chips, to identify and characterize temporal and tissue specific gene expression. When the unique sequences described in SEQ ID NOS:10-5,504 are expressed in mouse embryonic stem cells (“ES cells”), they provide a method of identifying phenotypic expression of the particular gene, as well as a method of assigning function to previously unknown genes. The unique sequences described in SEQ ID NOS:10-5,504 can be further used to identify the gene of interest from many sources, including, but not limited to, libraries consisting of cDNA or genomic clones, and for the in silico screening of nucleic acid and protein databases. The unique sequences described in SEQ ID NOS:10-5,504 have further utility for genetic manipulations such as antisense inhibition and gene targeting.

### 5.0 BRIEF DESCRIPTION OF THE FIGURES

FIG. 1A, FIG. 1B, FIG. 1C, and FIG. 1D. Illustration of a gene trap vector and methodology. FIG. 1A. Illustration of a retroviral vector that can be used to practice the described invention. FIG. 1B. Schematic of how a typical cellular genomic locus is effected by the integration of a retroviral construct into intron sequences of a cellular gene. FIG. 1C. Chimeric transcripts produced by the gene trap event, as well as locations of the binding sites for PCR primers. FIG. 1D. Schematic showing how PCR amplified cDNAs are directionally cloned into a suitable GTS vector.

FIG. 2. Block diagram that is exemplary of a computer system that can be used to implement the computer-based methods of the present invention.

### 6.0 Detailed Description of the Invention

The present invention is directed to novel human polynucleotide sequences obtained from cDNA libraries generated by the normalized expression of genomic exons using gene trap technology. In particular, the disclosed novel polynucleotides were generated using a modified reverse-orientation retroviral gene trap vector that was integrated nonspecifically into the target cell genome, although other polynucleotide (DNA or RNA) gene trap vectors could have been introduced to the target cells by, for example, transfection, electroporation, or retrotransposition. Retroviral vectors that can be used to practice the invention (as well as methods and recombinant tools for making and using the described GTSs) are disclosed in, inter alia, U.S. Pat. No. 6,436,707.

After integration, the exogenous promoter of the sequence acquisition, or 3′ gene trap, component of the vector was used to express and splice a chimeric mRNA that was subsequently reverse transcribed, amplified, and subject to DNA sequence analysis. Unlike conventional cDNA libraries, the presently disclosed libraries are largely unaffected by the bias inherent in cDNA libraries that rely solely on endogenous mRNA expression. Additionally, by integrating a vector into the target cell genes, a chimeric mRNA is produced that allows for the specific expansion and isolation of cDNAs corresponding to the chimeric mRNAs using vector specific primers.

As used herein, the term “gene trapped sequence” (“GTS”), refers to nucleotide sequences that correspond to naturally occurring endogenously encoded human exons that have been expressed as part of a chimeric “gene trapped” mRNA. The chimeric mRNA typically incorporates at least a portion of the sequence that is engineered into the sequence acquisition exon of a gene trap vector that, inter alia, facilitates cDNA production by reverse transcriptase and amplification of the cDNA by PCR to produce an isolated linear DNA molecule. The disclosed GTSs do not include vector encoded sequences.

The term “GTS” not only refers to polynucleotides that are exactly complementary to naturally occurring human mRNA, but also refers to “GTS derivatives”. The term GTS derivative refers to heterologs, paralogs, orthologs, and allelic variants of the specific GTSs described herein. A GTS may include the complete coding region for a naturally occurring peptide or polypeptide, or a complete open reading frame.

The term “GTS peptide”, as used herein, includes oligo- or polypeptides sharing biological activity and/or immunogenicity (or immunological cross-reactivity) with an amino acid sequence encoded by at least one of the disclosed GTSs, or the complement thereof. The terms “biological activity” (or “biological characteristics”) of a polypeptide refers to the structural or biochemical function of the polypeptide in the normal biological processes of the organism in which the polypeptide naturally occurs. Examples of such characteristics include protein structure and/or conformation, which can be determined biochemically with appropriate ligands or receptors, or by suitable biological assays.

A GTS peptide may also correspond to a full-length naturally occurring peptide or polypeptide. GTS peptides can have amino acid sequences that directly correspond to naturally occurring polypeptides or amino acid sequences, or can comprise minor variations. Such variations can include amino acid substitutions that are the result of the replacement of one amino acid with another amino acid having similar structural and/or chemical properties, such as the substitution of a leucine with an isoleucine or valine, an aspartate with a glutamate, or a threonine with a serine, i.e., conservative amino acid replacements. Additional variations include minor amino acid deletions and/or insertions, typically in the range of about 1 to 6 amino acids, and can also include one or more amino acid substitutions. Guidance in determining which GTS peptide amino acid residues can be replaced or deleted without abolishing the biological activity of interest may be determined empirically, or by using computer amino acid sequence databases to identify polypeptides that are homologous to a given GTS peptide, and trying to avoid amino acid substitutions in conserved regions of homology.

“Homology” refers to the similarity or the degree of similarity between a reference, or known, polynucleotide and/or polypeptide and a test nucleotide sequence and/or its corresponding amino acid sequence. As used herein, “homology” is defined by sequence similarity between a reference sequence and at least a portion of the newly sequenced nucleotide. Usually, a corresponding amino acid sequence similarity exists between the peptides encoded by such homologous sequences.

To determine whether proteins are homologous, the GTS sequence is translated into the corresponding amino acid sequence. The amino acid sequence is then compared with reference polypeptide sequences. A short string of matching amino acid sequence can constitute good evidence of homology (for example, repeating Gly-Pro-X sequence, or the presence of an RGD motif). However, typically a larger number of similar amino acids is required to label two sequences homologous. Generally, the match needs to be at least about 7 or 8 amino acids, among which perhaps one mismatch is allowed. These criteria allow good sensitivity in finding all relevant sequences while providing a threshold amount of selectivity.

After peptide homology has been found, the respective nucleotide sequences are compared. An alignment of the reference and new sequences should show at least about 60%, and preferably at least about 65%, agreement over the minimum of 21 nucleotides that correspond to the 6 matching amino acids. Generally, a low percentage of agreement is acceptable if the differences are in the “wobble” position (or third nucleotide of the triplet coding for an amino acid).

As used herein, a “mutated” polypeptide has an altered primary structure typically resulting from corresponding mutations in the nucleotide sequence encoding the protein or polypeptide. As such, the term “mutated” polypeptides can include allelic variants. Mutational changes in the primary structure of a polypeptide generally result from deletions, additions or substitutions. A “deletion” is defined as a change in a polypeptide sequence in which one or more internal amino acid residues are absent. An “addition” is defined as a change in a polypeptide sequence in which one or more internal amino acid residues have been added compared to the wild-type. A “substitution” results from replacement of one or more amino acid residues by other residues. A polypeptide “fragment” is a polypeptide consisting of a primary amino acid sequence that is identical to a portion of the primary sequence of the polypeptide to which the polypeptide is related.

A host cell “expresses” a gene or DNA when the gene or DNA is transcribed into RNA that may optionally be translated to produce a polypeptide.

“Recombinant” means the GTS is adjacent to “backbone” nucleic acids to which it is not adjacent in its natural environment. To be “enriched”, the GTS's will represent 5% or more of the number of nucleic acid inserts in a population of expression vectors, self-replicating nucleic acids, viruses, integrating nucleic acids, and other vectors or nucleic acids used to maintain or manipulate a GTS. Preferably, the enriched GTS represent 15% or more of the number of nucleic acid inserts in the population of recombinant backbone molecules. In a highly preferred embodiment, the enriched GTSs represent 90% or more of the number of nucleic acid inserts in the population of recombinant molecules. Additional definitions exemplary of the general level of skill in the art can be found in U.S. Pat. No. 6,528,289.

The subject invention also includes GTSs that are incorporated into expression vectors and transformed into host cells, which in certain embodiments subsequently express the polynucleotides and/or polypeptides encoded by the GTSs.

The subject invention also includes antibodies capable of selectively binding to GTS peptides, as well as methods of detecting a GTS peptide or the corresponding protein by combining a sample for analysis with an antibody capable of selectively binding to a GTS peptide, and detecting the formation of antibody complexes present in the sample.

The subject invention also includes a method of isolating a GTS peptide, or its corresponding protein, comprising the step of separating the GTS peptide or protein from a solution utilizing an antibody capable of selectively binding to the GTS peptide or its corresponding protein.

The subject invention also provides for markers for use in detecting diseases, biological events, cell-types, and tissues, which comprise at least a portion of a GTS sequence.

Further, the subject invention provides polynucleotide markers useful for physical and genetic mapping of the human, and/or certain model organism, genome(s). In particular, the nucleotide sequences in the Sequence Listing provide sequence tagged sites (STS) that will be useful in completing an STS-based physical map of the human genome, which is a goal of the human genome project (Collins and Galas, Science 262:43-46, 1993). Additionally, some of these sequences will identify new genes. These new genes will be useful in completing physical and genetic maps of all the genes in the human genome, another goal of the human genome project.

The exons contained in the disclosed GTSs contain open reading frames (present in one of the three reading frames in either orientation of the sequence). Typically, the gene trap strategy employed to generate the GTS sequences allows for the directional cloning and identification of the sense strand. However, it is possible that occasional sequencing errors, or random reverse transcription or PCR aberrations, will mask the presence of the appropriate open reading frame (ORF). In such cases, it is possible to determine the corresponding GTS sequence by expressing the GTS in an appropriate expression system and determining the amino acid sequence by standard peptide mapping and sequencing techniques (see, e.g., “Current Protocols in Molecular Biology”, Vol. 2, Ch. 16 (Ausubel et al., eds., Green Publishing Associates, Inc., and John Wiley & Sons, Inc., New York, 1989)). Additionally, the actual reading frame and amino acid sequence of a given nucleotide sequence may be determined by in vitro synthesis of a portion of an oligopeptide comprising a possible amino acid sequence, and preparing antibodies to the oligopeptide. If the antibodies react with cells from which the GTS of interest was derived, the reading frame is likely correct. Alternatively, codon usage analysis can be used to track and correct reading frame shifts in gene sequence data.

The correct amino acid sequence of a GTS protein is largely a function of the DNA sequence, and the correct amino acid sequence can be readily determined using routine techniques. For example, by providing independent three fold sequencing coverage of the GTS library, random sequencing, reverse transcriptase (RT), or PCR errors can be identified and corrected by selecting the sequence represented by the majority of gene trap sequences covering a given nucleotide.

The nucleotide sequences of the Sequence Listing may contain some sequencing errors, and several may contain nucleotides that have not been precisely identified, typically designated by an N, rather than A, T, C, or G. Since each of the nucleotide sequences in the Sequence Listing is believed to uniquely identify a novel GTS, any sequencing errors or N's do not present a problem in practicing the subject invention. Several methods employing standard recombinant methodology, for example, as described in “Molecular Cloning, A Laboratory Manual” (Sambrook et al., eds., Cold Spring Harbor Press, Cold Spring Harbor, N.Y., 1989), and periodic updates thereof, may be used to correct errors and complete the missing sequence information. For example, a nucleotide and/or oligonucleotide corresponding to a portion of a nucleotide sequence of a GTS of interest can be chemically or biochemically synthesized in vitro, and used as a-hybridization probe to screen a cDNA library to identify and obtain library isolates comprising recombinant DNA sequences containing the corresponding GTS cDNA sequence. The library isolate can be subjected to nucleotide sequencing, using standard sequencing procedures, to obtain a complete and accurate nucleotide sequence.

For the purposes of this disclosure, the term “isolated” or “purified” polynucleotide comprises a polynucleotide purified from a natural cell or tissue, as well as polynucleotides that are complementary to the polynucleotides isolated from the natural cell or tissue. One example of an isolated or purified polynucleotide, or a substantially isolated preparation thereof, is a preparation in which the polynucleotide of interest represents at least about 80 percent, at least about 85 percent, or at least about 90 to 95 percent or more, of the net product(s) that can be visualized on a DNA agarose gel stained with ethidium bromide.

The described GTSs were obtained from isolates of a cDNA library. Clones isolated from cDNA libraries generated by 3′ gene trapping typically contain only a portion of the mature RNA transcript that has been spliced to a vector-encoded sequence acquisition exon, and therefore such clones may only encode a portion of the polypeptide of interest (however, it should be appreciated that a number of the disclosed GTSs may encode full-length ORFs). To obtain the remainder of the sequence, the GTSs can be used as hybridization probes to screen the same or a different cDNA library, and additional clones identified can be purified and characterized using standard methods (Benton and Davis, Science 196:180-182, 1977). Once sufficiently purified, the size of the DNA insert can be approximated by agarose gel electrophoresis, and the larger clones can be analyzed to determine the exact number of bases by DNA sequencing. Frequently, the use of a library different from the one that contained the original clone is useful for this purpose, for example a library that has been prepared with extra care to extend cDNA synthesis to full-length, or a library that has been intentionally primed with random primers in order to “jump over” particularly difficult regions of the transcript sequence.

Missing upstream DNA sequence can also be obtained by “primer extension” of the cDNA isolate, a practice common in the art (“Molecular Cloning: Laboratory Manual”, supra, Vol. 2 at pg. 7.79-7.83), whereby a sequence-specific oligonucleotide is used to prime reverse-transcription near the 5′-end of the cDNA clone, and the resulting product is either cloned into a bacterial vector or is analyzed directly by DNA sequencing. Additionally, newer methods to extend clones in either direction employ oligonucleotide-directed thermocyclic DNA amplification of the missing sequences, wherein a combination of a cDNA-specific primer and a degenerate vector-specific, or oligo-dT-binding, second oligonucleotide are used to prime strand synthesis. In any of the above methods, or other methods of detecting additional cDNA sequence, two or more resulting clones containing the partial cDNA sequence can be recombined to form a single full-length cDNA by standard cloning methods. The resulting full-length cDNA may subsequently be transferred into any of a number of appropriate expression vectors, as described herein.

In many instances, the sequencing of clones resulting from independent nonspecific gene trap events will result in a natural redundancy (sequencing more than one cDNA from a particular gene). As discussed above, this feature is a built-in form of error detection and correction. These independent gene trap events can also be combined using any overlapping regions of sequence into a larger contiguous sequence (“contig”), that may contain the complete nucleotide sequence of the full length cDNA. Similar methodology can be used to combine one or more GTSs with one or more publicly available and/or proprietary ESTs to synthesize, electronically or chemically, a contiguous sequence.

The ABI Assembler application, part of the INHERITS DNA analysis system (Applied Biosystems, Inc., Foster City, Calif.), creates and manages sequence assembly projects by assembling data from selected sequence fragments into a larger sequence. The Assembler combines two advanced computer technologies that maximize the ability to assemble sequenced DNA fragments into Assemblages, a special grouping of data where relationships between sequences are shown by graphic overlap, alignment, and statistical views. The process is based on the Meyers-Kececioglu model of fragment assembly (INHERITS™ Assembler User's Manual; Applied Biosystems), and uses graph theory as the foundation of a very rigorous multiple sequence alignment program for assembling DNA sequence fragments. Additional methods of analyzing and using partial length sequences, such as most GTSs, and obtaining full-length versions thereof, are discussed in U.S. Pat. Nos. 5,817,479 and 5,552,281, and U.S. patent application Ser. No. 08/904,468.

It will be appreciated by those skilled in the art that as a result of the degeneracy of the genetic code (see, e.g., “Molecular Cell Biology”, Table 4-1 at page 109 (Darnell et al., eds., Scientific American Books, New York, N.Y., 1986)), a multitude of GTS nucleotide sequences, some bearing minimal nucleotide sequence homology to the nucleotide sequence of genes naturally encoding GTS peptides, can be produced. The invention has specifically contemplated each and every possible variation of nucleotide sequence that could be made by selecting combinations based on possible codon choices. These combinations are made in accordance with the standard triplet genetic code as applied to the nucleotide sequence of naturally occurring human GTS nucleotide sequences, and all such variations are to be considered as being specifically disclosed. Once the triplet codons are “translated” (which can be done electronically) into their amino acid counterparts, the amino acid sequences encoded by the GTS ORFs effectively constitute a generic representation of the various nucleotide sequences that can encode the amino acid sequence (i.e., each amino acid is generic for the various nucleotide codons that correspond to that amino acid).

The presently described novel human GTSs provide unique tools for diagnostic gene expression analysis, for cross-species hybridization analysis, and for genetic manipulations using a variety of techniques (including, but not limited to, antisense inhibition, gene targeting, identification or generation of full-length cDNA, mapping the human genome, gene therapy, and/or gene delivery). Furthermore, the expression-based detection and isolation of the described novel polynucleotides verifies that the genes encoding these sequences have not been inactivated by, for example, the covalent modification (methylation, acetylation, glycosylation, etc.) of the target cell genome, or inhibiting the function of transcriptional control elements. The fact that the genes have not been inactivated in the target cell genome can indicate an involvement in cellular metabolism, catabolism, homeostasis, or any of a wide variety of developmental and cell differentiation processes or the regulation of physiological or endocrine functions in the body (although treating the target cell with, for example, histone deacetylators can partially compensate for such inactivation and expand the target size of a given trapping construct). These data are especially useful when correlated with cDNA data from differentiated tissues, cells, and/or cell lines, in order to determine whether the absence of expression is regulated at the level of transcription or gene inactivation.

### 6.1 Polynucleotides of the Present Invention

The nucleotide sequences of the various isolated human GTSs of the present invention appear in the Sequence Listing as SEQ ID NOS:10-5,504. Additional embodiments of the present invention are GTS variants, or homologs, paralogs, orthologs, etc., which include isolated polynucleotides, or complements thereof, that hybridize to one or more of the disclosed GTSs of SEQ ID NOS:10-5,504 under stringent, or preferably highly stringent, conditions. By way of example and not limitation, high stringency hybridization conditions can be defined as follows. Prehybridization of filters containing DNA to be screened is carried out from 8 hours to overnight at 65° C. in a buffer containing 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe (alternatively, as in all hybridizations described herein, approximately 42, 44, 46, 48, 50, 52, 54, 56, 58, 62, 64, 66, 68, 70, or about 72 degrees or more can be used, with a suitable concentration of salt). The filters are then washed in approximately 1× wash mix (10× wash mix contains 3 M NaCl, 0.6 M Tris base, and 0.02 M EDTA, alternatively, as with all washes described herein, 2×, 3×, 4×, 5×, 6× wash mix, or more, can be used) twice for 5 minutes each at room temperature, then in 1× wash mix containing 1% SDS at 60° C. (alternatively, as in all washes described herein, approximately 42, 44, 46, 48, 50, 52, 54, 56, 58, 62, 64, 66, 68, 70, or about 72 degrees or more can be used) for about 30 minutes, and finally in 0.3× wash mix (alternatively, as in all final washes described herein, approximately, 0.2×, 0.4×, 0.6×, 0.8×, 1×, or any concentration between about 2× and about 6× can be used in conjunction with a suitable wash temperature) containing 0.1% SDS at 60° C. (alternatively, as in all final washes described herein, approximately 42, 44, 46, 48, 50, 52, 54, 56, 58, 62, 64, 66, 68, 70, or about 72 degrees or more can be used) for about 30 minutes. The filters are then air dried and exposed to x-ray film for autoradiography. In an alternative protocol, washing of filters is done at 37° C. for 1 hour in a solution containing 2×SSC, 0.01% PVP, 0.01% Ficoll, and 0.01% BSA. This is followed by a wash in 0.1×SSC at 50° C. for 45 minutes before autoradiography. Another example of hybridization under highly stringent conditions is hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (see, e.g., “Current Protocols in Molecular Biology”, supra, at p. 2.10.3).

Preferably, such GTS variants will encode at least a portion or domain of a, preferably naturally occurring, protein or polypeptide that encodes a functional equivalent to a protein or polypeptide, or portion or domain thereof, encoded by the disclosed GTSs. Additional examples of GTS variants include polynucleotides, or complements thereof, that are capable of binding to the disclosed GTSs under less stringent conditions, such as moderately stringent conditions, (e.g., washing in 0.2×SSC/0.1% SDS at 42° C. (see, e.g., “Current Protocols in Molecular Biology”, supra)). Moderately stringent conditions can be additionally defined, for example, as follows. Filters containing DNA are pretreated for 6 hours at 55° C. in a solution containing 6×SSC, 5× Denhart's solution, 0.5% SDS and 100 μg/ml denatured salmon sperm DNA. Hybridizations are carried out in the same solution, and 5-20×10⁶ cpm ³²P-labeled probe is used. Filters are incubated in hybridization mixture for 18-20 hours at 55° C. The filters are then washed in approximately 1× wash mix twice for 5 minutes each at room temperature, then in 1× wash mix containing 1% SDS at 60° C. for about 30 minutes, and finally in 0.3× wash mix containing 0.1% SDS at 60° C. for about 30 minutes. The filters are then air dried and exposed to x-ray film for autoradiography. In an alternative protocol, washing of filters is done twice for 30 minutes at 60° C. in a solution containing 1×SSC and 0.1% SDS. Filters are blotted dry and exposed for autoradiography.

Other conditions of moderate stringency that may be used are well-known in the art. For example, washing of filters can be done at 37° C. for 1 hour in a solution containing 2×SSC and 0.1% SDS. Such less stringent conditions may also be, for example, low stringency hybridization conditions (e.g., as employed for cross-species hybridizations; see, e.g., Shilo and Weinberg, Proc. Natl. Acad. Sci. USA 78:6789-6792, 1981). By way of example, and not limitation, a procedure using low stringency conditions is as follows. Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridization is carried out in a solution containing 35% formamide, 5×SSC, 50 mM Tris-HCl (pH 7.5), 5 mM EDTA, 0.02% PVP, 0.02% Ficoll, 0.2% BSA, 100 μg/ml salmon sperm DNA, 10% (wt/vol) dextran sulfate, and 5-20×10⁶ cpm of ³²P-labeled probe is used. Filters are incubated in hybridization mixture for 18-20 hours at 40° C. The filters are then washed in approximately 1× wash mix twice for five minutes each at room temperature, then in 1× wash mix containing 1% SDS at 60° C. for about 30 minutes, and finally in 0.3× wash mix containing 0.1% SDS at 60° C. for about 30 minutes. The filters are then air dried and exposed to x-ray film for autoradiography. In an alternative protocol, washing of filters is done for 1.5 hours at 55° C. in a solution containing 2×SSC, 25 mM Tris-HCl (pH 7.4), 5 mM EDTA, and 0.1% SDS. The wash solution is replaced with fresh solution, and incubated an additional 1.5 hours at 60° C. Filters are then blotted dry and exposed for autoradiography. If necessary, filters can be washed for a third time at 65-68° C., and re-exposed to film. Preferably, GTS variants identified or isolated using any of the above methods will encode a functionally equivalent gene product (i.e., a protein, polypeptide, or domain thereof, associated with at least one of the functions and/or structures encoded by the complementary GTS, or a fragment thereof).

Additionally contemplated are gene products, or their functional equivalent, encoded by a polynucleotide sequence that is at least about 99, 95, 90, or about 85 percent similar or identical to a GTS described in the Sequence Listing, or a fragment thereof (as measured by BLAST sequence comparison analysis using, for example, the University of Wisconsin GCG sequence analysis-package (SEQUENCHER 3.0, Gene Codes Corp., Ann Arbor, Mich.) using standard default parameters).

Additional embodiments contemplated by the present invention include any polynucleotide sequence comprising a continuous stretch of nucleotide sequence of at least 8, at least 10, at least 14, at least 20, at least 30, at least 40, or at least 60 consecutive nucleotides originally disclosed in, or otherwise unique to, any of the GTSs of SEQ ID NOS:10-5,504, up to a contiguous stretch of about several hundred bases, or an entire GTS sequence. Functional equivalents of the gene products of SEQ ID NOS:10-5,504 include naturally occurring variants of SEQ ID NOS:10-5,504 present in other species, and mutant variants, both naturally occurring and engineered, that retain at least one functional activity of the gene products of SEQ ID NOS:10-5,504.

The present invention also contemplates nucleic acid sequences comprising at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, or 350 or more contiguous nucleotides from any of the disclosed GTS sequences. Additionally, the invention contemplates nucleic acid sequences comprising polynucleotides encoding at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, or 100 or more contiguous amino acids encoded by any of the described GTSs.

The invention also includes degenerate variants of the disclosed GTS sequences, and products encoded thereby. The invention further includes GTS derivatives wherein a GTS sequence, or variant thereof, is operably linked to another polynucleotide molecule. The linkage may be direct, or through another polynucleotide of any sequence and of a length of about 1,000 base pairs, about 500 base pairs, about 300 base pairs, about 200 base pairs, about 150 base pairs, about 100 base pairs, or about 50 base pairs, or less.

The invention also includes polynucleotide molecules, including DNA, that hybridize to, and are therefore the complements of, the disclosed GTS nucleotide sequences. Such hybridization conditions may be highly stringent or less highly stringent, as described herein. Where the nucleic acid molecules are deoxyoligonucleotides (“DNA oligos”), highly stringent conditions may refer to, for example, washing in 6×SSC/0.05% sodium pyrophosphate at 37° C. (for 14-base DNA oligos), 48° C. (for 17-base DNA oligos), 55° C. (for 20-base DNA oligos), or 60° C. (for 23-base DNA oligos). Similar conditions are contemplated for RNA oligos corresponding to a portion of any of the disclosed GTS sequences.

These nucleic acid molecules may encode or act as antisense molecules to polynucleotides comprising at least a portion of any of the sequences shown in SEQ ID NOS:10-5,504. Such molecules are useful, for example, to regulate the expression of genes comprising a nucleotide sequence of any of SEQ ID NOS:10-5,504, and can also be used, for example, as antisense primers in amplification reactions of gene sequences. With respect to gene regulation, such techniques can be used to regulate, for example, developmental processes by modulating the expression of genes in embryonic stem cells. Further, such sequences may be used as part of ribozyme and/or triple helix sequences that can be used to regulate gene expression. Still further, such molecules may be used as components of diagnostic methods whereby, for example, the presence of a particular allele of a gene that contains any of the sequences of SEQ ID NOS:10-5,504 may be detected. Of particular interest is the use of the disclosed GTSs to conduct analysis of single nucleotide polymorphisms (SNPs), and particularly coding region SNPs or “cSNPs”, in the human genome, or as general or individual-specific forensic markers. When so applied, a collection of GTSs is obtained from an individual, and screened against a control database of cSNPs (or other genetic markers) that have previously been associated with disease, suitability or susceptibility (or sensitivity) to specific drugs or therapies, or virtually any other human trait that correlates with a given cSNP or genetic marker, or assortment thereof. Additionally, the described GTSs are useful as genetic markers for the prenatal analysis of congenital traits or defects.

In addition to the nucleotide sequences described herein, full-length cDNA or gene sequences that contain any of SEQ ID NOS:10-5,504, present in humans and/or homologs of any of those genes present in other species, can be identified and isolated using standard molecular biological techniques. For example, oligonucleotides corresponding to either the 5′ or 3′ terminus of the cDNA sequence may be used to obtain longer nucleotide sequences. In order to clone a full-length cDNA sequence, or a variant or homolog, from any species, labeled DNA probes made from nucleic acid fragments corresponding to any portion of the cDNA sequences disclosed herein may be used to screen a cDNA library. For example, a cDNA library can be plated out to yield a maximum of about 30,000 pfu for each 150 mm plate, and approximately 40 plates may be screened. The plates are incubated at 37° C. until the plaques reach a diameter of about 0.25 mm, or are just beginning to make contact with one another (3-8 hours). Nylon filters are placed onto the soft top agarose, and after 60 seconds, the filters are peeled off and floated on a DNA denaturing solution (0.4 N sodium hydroxide) for 1-5 minutes. Next, the filters are immersed in neutralizing solution (1 M Tris-HCl, pH 7.5), before being allowed to air dry. The filters are prehybridized in casein hybridization buffer (10% dextran sulfate, 0.5 M NaCl, 50 mM Tris-HCl, pH 7.5, 0.1% sodium pyrophosphate, 1% casein, 1% SDS, 0.5 mg/ml denatured salmon sperm DNA) for 6 hours at 60° C. The labeled probe is then denatured by heating to 95° C. for 2 minutes, and then added to the prehybridization solution containing the filters. The filters are hybridized at 60° C. for about 16 hours. The filters are then washed in approximately 1× wash mix twice for 5 minutes each at room temperature, then in 1× wash mix containing 1% SDS at 60° C. for about 30 minutes, and finally in 0.3× wash mix containing 0.1% SDS at 60° C. for about 30 minutes. The filters are then air dried and exposed to x-ray film for autoradiography. After developing, the film is aligned with the filters to select positive plaques. If a single, isolated positive plaque cannot be obtained, an agar plug surrounding the plaque is removed and placed in lambda dilution buffer (0.1 M NaCl, 0.01 M magnesium sulfate, 0.035 M Tris-HCl, pH 7.5, 0.01% gelatin), and again plated and screened to obtain single, well isolated positive plaques. Positive plaques are isolated, and the cDNA clones sequenced using primers based on the known cDNA sequence. This step may be repeated until a full-length cDNA is obtained.

It may be necessary to screen multiple cDNA libraries from different sources/tissues to obtain a full-length cDNA. In the event that it is difficult to identify cDNA clones encoding the complete 5′ terminal coding region, a common situation in cDNA cloning, the RACE (Rapid Amplification of cDNA Ends) technique may be used. RACE is a proven PCR-based strategy for amplifying the 5′ end of incomplete cDNAs. 5′-RACE-Ready cDNA synthesized from human fetal liver containing a unique anchor sequence is commercially available (Clontech, Palo Alto, Calif.). To obtain the 5′ end of the cDNA, PCR is carried out, for example, on 5′-RACE-Ready cDNA using the provided anchor primer and the 3′ primer. A secondary PCR reaction is then carried out using the anchored primer and a nested 3′ primer according to the manufacturer's instructions. Once obtained, the full-length cDNA sequence may be translated into amino acid sequence, and examined for certain landmarks found in, or any structural similarities to, the amino acid sequences encoded by SEQ ID NOS:10-5,504.

The identification of homologs, heterologs, or paralogs of SEQ ID NOS:10-5,504 in other, preferably related, species can be useful for developing additional animal model systems that are closely related to humans for purposes of drug discovery. Genes at other genetic loci within the genome that encode proteins that have extensive homology to one or more domains of the gene products encoded by SEQ ID NOS:10-5,504, as well as clones derived from alternatively spliced transcripts in the same or different species, can be identified, for example, by screening cDNA libraries using filter hybridization with duplicate filters and a labeled probe. The labeled probe can, in certain embodiments, contain at least 15-30 contiguous nucleotides from the nucleotide sequence presented in any of SEQ ID NOS:10-5,504. The hybridization and washing conditions should be of a lower stringency when the cDNA library is derived from a different, or heterologous, organism. With respect to the cloning of a mammalian homolog, heterolog, ortholog, or paralog, using a probe derived from any of the disclosed cDNA sequences, hybridization can, for example, be performed at 65° C. overnight in Church's buffer (7% SDS, 250 mM NaHPO₄, 2 mM EDTA, 1% BSA). Washes can be done with 2×SSC and 0.1% SDS at 65° C., and then with 0.1×SSC and 0.1% SDS at 65° C. Additional low stringency conditions are well-known to those of skill in the art (see, e.g., “Molecular Cloning, A Laboratory Manual”, supra, and “Current Protocols in Molecular Biology”, supra), and will vary predictably depending on the specific organism from which the cDNA library is derived.

Alternatively, a labeled nucleotide probe corresponding to any of SEQ ID NOS:10-5,504 may be used to screen a genomic library derived from an organism of interest, again, using appropriately stringent conditions. The identification and characterization of human genomic clones is helpful for designing diagnostic tests and clinical protocols for treating disorders, for example in human patients that are known to have or suspected of having a developmental or cell differentiation disorder or abnormality. For example, sequences derived from regions adjacent to the intron/exon boundaries of the human gene can be used to design primers for use in diagnostic amplification assays to detect mutations within the exons, introns, and/or splice sites (e.g., splice acceptor and/or donor sites).

Alternatively, homologs can be isolated from nucleic acid of an organism of interest by performing PCR using two oligonucleotide primers derived from SEQ ID NOS:10-5,504, or two degenerate oligonucleotide primer pools designed on the basis of amino acid sequences within the gene products encoded by SEQ ID NOS:10-5,504. The template for the reaction may be cDNA obtained by reverse transcription of mRNA prepared from, for example, human or non-human cell lines, cell-types, or tissues from the organism of interest, including, but not limited to, ES cells. The PCR product may be sequenced directly, or subcloned and sequenced, to ensure that the amplified sequence represents sequence from the gene of interest, corresponding any one of SEQ ID NOS:10-5,504. The PCR fragment may then be used to isolate a full-length cDNA clone, using any of a number of standard methods. For example, the amplified fragment may be labeled and used to screen a cDNA library, such as a bacteriophage cDNA library. Alternatively, the labeled fragment may be used to isolate genomic clones via the screening of a genomic library.

PCR can also be utilized to isolate full-length cDNA sequences, using any standard cloning strategy (see, e.g., “Molecular Cloning, A Laboratory Manual”, supra). For example, RNA may be isolated, using any standard procedure, from an appropriate cellular source (i.e., one known to express, or suspected of expressing, the gene of interest corresponding to any of SEQ ID NOS:10-5,504), such as ES cells. A RT reaction can be performed on the RNA using an oligonucleotide primer specific for a sequence near the 5′ end of any of SEQ ID NOS:10-5,504, for the priming of first strand synthesis. The resulting RNA/DNA hybrid can be “tailed” with guanines, for example, using a standard terminal transferase reaction, digested with RNase H, and second strand synthesis primed with a poly-C primer. Thus, cDNA sequences upstream from any of SEQ ID NOS:10-5,504 can easily be isolated. Alternatively, a cDNA or genomic library can be screened using 5′ PCR primer that hybridizes to vector sequence, and a 3′ PCR primer corresponding to any of SEQ ID NOS:10-5,504.

The sequence of a gene corresponding to any of the sequences of SEQ ID NOS:10-5,504 can also be used to isolate mutant alleles of that gene. Such mutant alleles may be isolated from an individual either known to have or suspected of having a genotype that contributes to a developmental and/or cell differentiation or proliferation disorder or abnormality, or any other particular disease of interest. Mutant alleles and mutant allele products may then be utilized in the therapeutic and diagnostic programs described herein.

A mutant allele corresponding to any of SEQ ID NOS:10-5,504 can be isolated from a genomic library made using DNA obtained from one or more individuals suspected of carrying, or known to carry, the mutant gene. The corresponding normal gene, or any suitable fragment thereof, can be labeled and used as a probe to identify the corresponding mutant gene in such a library. Clones containing the mutant gene sequences may then be identified and analyzed by DNA sequence analysis. By comparing the DNA sequence of the mutant gene to that of the normal gene, the mutation(s) responsible for the loss or alteration of function of the mutant gene product can be ascertained. Additionally, such sequences can be used to detect gene regulatory (e.g., promoter or promoter/enhancer) defects, which can affect development and/or cell differentiation.

Additionally, a cDNA of a mutant gene corresponding to any of the sequences of SEQ ID NOS:10-5,504 can be isolated as discussed above, or, for example, by using PCR. In this method, the first cDNA strand may be synthesized by hybridizing an oligo-dT oligonucleotide to mRNA isolated from cells derived from an individual known to carry, or suspected of carrying, the mutant gene, and extending the new strand with reverse transcriptase. The second strand of the cDNA is then synthesized using an oligonucleotide that hybridizes specifically to the 5′ region of the normal gene. The amplified product can be sequenced directly, or cloned into a suitable vector and subsequently sequenced. When compared to the sequence of the normal cDNA, the mutation(s) responsible for the loss or alteration of function of the mutant gene product can be identified.

Alternatively, mRNA from cell-types known to express, or suspected of expressing, such mutant alleles can be used to construct a mutant cDNA library. The library can be screened using a labeled probe corresponding to the normal gene, or any suitable fragment thereof, to identify the corresponding mutant allele. Clones containing the mutant gene sequences may then be identified and analyzed by DNA sequence analysis. Once again, by comparing the sequence of the mutant allele to the sequence of the normal cDNA, the mutation(s) responsible for the loss or alteration of function of the mutant gene product can be identified.

Furthermore, mRNA from cell-types known to express, or suspected of expressing, such mutant alleles can be used to construct a protein expression library. In this manner, gene products made by the putatively mutant cell-type may be expressed and screened using standard antibody screening techniques in conjunction with antibodies raised against the corresponding normal gene product, or a portion thereof, as described below in Section 6.4 (for screening techniques, see, e.g., “Antibodies: A Laboratory Manual” (Harlow and Lane, eds., Cold Spring Harbor Press, Cold Spring Harbor, N.Y., 1988)). Screening can also be accomplished using a labeled fusion protein. In cases where a mutation results in an expressed gene product with altered function (e.g., as a result of a missense or frame shift mutation), polyclonal antibodies to the normal gene product are likely to cross-react with the mutant gene product. Library clones detected via their reaction with such labeled antibodies or fusion proteins can be purified and subjected to sequence analysis according to methods well-known to those of skill in the art.

The invention also encompasses nucleotide sequences that encode mutant isoforms of any of the amino acid sequences encoded by the GTSs of SEQ ID NOS:10-5,504, peptide fragments and truncated versions thereof, and fusion proteins including any of the above. Examples of such fusion proteins include, but are not limited to, an epitope tag that aids in detection or purification of the fusion protein, or fluorescent protein, luminescent protein, or enzyme, that can be used as a marker.

The present invention also encompasses: (a) RNA or DNA vectors that contain any portion of any one or more of SEQ ID NOS:10-5,504, and/or their complements, as well as any sequence that encodes any of the peptides or proteins encoded thereby; (b) DNA vectors that contain a cDNA that spans substantially the entire ORF corresponding to any of SEQ ID NOS:10-5,504 and/or their complements; (c) DNA expression vectors comprising any of the foregoing sequences, or portion thereof, operatively associated with a regulatory element that directs the expression of the coding sequences in a host cell; and (d) genetically engineered host cells that contain a cDNA that encodes the entire coding sequence, or portion thereof, corresponding to any of the sequences of SEQ ID NOS:10-5,504, operatively associated with a regulatory element that directs the expression of the coding sequence in the host cell.

Such regulatory elements are generally recombinantly positioned either in vivo (such as in gene activation) or in vitro. As used herein, regulatory elements include, but are not limited to, inducible and non-inducible promoters, enhancers, operators, and other elements known to those skilled in the art that drive and regulate expression. Such regulatory elements include, but are not limited to, the human cytomegalovirus (hCMV) immediate early gene, regulatable viral elements (particularly retroviral LTR promoters), the early or late promoters of SV40 adenovirus, the lac system, the trp system, the TAC system, the TRC system, the major operator and promoter regions of phage lambda, the control regions of fd coat protein, the promoter for 3-phosphoglycerate kinase (PGK), the promoters of acid phosphatase, and the promoters of the yeast alpha-mating factors.

### 6.2 Proteins and Polypeptides Encoded by Polynucleotides Expressed in Modified Human Cells

The present invention also encompasses peptides and proteins encoded by the open reading frame of any of SEQ ID NOS:10-5,504, polypeptide and peptide fragments, mutated, truncated or deleted forms of those peptides and proteins, and fusion proteins containing any of those peptides and proteins. Such compositions can be prepared for a variety of uses, including, but not limited to, for generation of antibodies, as reagents in diagnostic assays, for identification of other cellular gene products involved in the regulation of development and/or cellular differentiation of various cell-types (for example ES cells), as reagents in screening assays for compounds that can be used in the treatment of disorders affecting development and/or cell differentiation, and as pharmaceutical reagents useful in the treatment of disorders affecting development and/or cell differentiation.

The invention also encompasses proteins, peptides, and polypeptides that are functionally equivalent to those encoded by SEQ ID NOS:10-5,504. Such functionally equivalent products include, but are not limited to, additions or substitutions of amino acid residues within an amino acid sequence encoded by SEQ ID NOS:10-5,504, but that result in a silent change, thus producing a functionally equivalent gene product. Amino acid substitutions can be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine, polar neutral amino acids include glycine, serine, threonine, cysteine, tyrosine, asparagine, and glutamine, positively charged (basic) amino acids include arginine, lysine, and histidine, and negatively charged (acidic) amino acids include aspartic acid and glutamic acid.

Random mutations or site-directed mutations can be introduced into DNA encoding the gene products of the current invention, using mutagenesis techniques well-known to the skilled artisan. The resulting gene products can be tested for activity to identify mutant peptides and proteins having similar, or in some cases increased, functionality. For example, the amino acid sequence of the gene products of the current invention can be aligned with the sequence of homologs from one or more different species. Mutant gene products can be engineered so that regions of interspecies identity are maintained, while the variable residues are altered, e.g., by deletion or insertion of one or more amino acid residue(s), or by substitution of one or more different amino acid residues. Conservative alterations at variable positions can generally be engineered in order to produce a mutant gene product that retains function, while non-conservative changes can generally be engineered at these variable positions to produce a mutant peptide or protein having an altered function. Alternatively, where alteration of function is desired, deletion, insertion, or non-conservative alterations in the conserved regions can be engineered. One of skill in the art may readily test such mutant peptides or proteins for any alteration in function using the teachings presented herein.

Mutations of DNA encoding the instant gene products can also be made to generate peptides and proteins that are better suited for expression, scale up, etc., in the particular host cell chosen for expression. For example, the triplet code for each amino acid can be modified to conform more closely to the preferential codon usage of the translational machinery of the particular host cell (for instance, to yield a messenger RNA molecule with a longer half-life). Those skilled in the art understand what modifications of the nucleotide sequence would conform the nucleotide sequence to preferential codon usage of the particular host cell, and/or to make the messenger RNA more stable. Such information is obtainable, for example, through use of computer programs, review of available research data on codon usage and messenger RNA stability, and other means known to those of skill in the art.

Additionally, the instant human polynucleotide sequences can be used in the molecular mutagenesis/evolution of proteins that are at least partially encoded by the instant GTSs using, for example, polynucleotide shuffling or related methodologies (see, e.g., U.S. Pat. Nos. 5,830,721 and 5,837,458).

As described above, the novel GTSs of the present invention encode novel ORFs. Such ORFs can occur in any of the six possible reading frames inherently present in the described GTSs. Such novel sequences can be used as, for example, epitope tags, or for the generation of hybrid/fusion proteins. Accordingly, another aspect of the present invention includes isolated polynucleotides comprising a sequence first disclosed in SEQ ID NOS:10-5,504 that encodes a novel peptide sequence, and the resulting fusion products.

As detailed above, fusion proteins in which a protein, polypeptide, or peptide of the present invention is fused to an unrelated protein are also within the scope of the invention. Such fusion proteins can be designed by those of skill in the art on the basis of experimental or functional considerations, and include, but are not limited to, fusion to an epitope tag, which can aid isolation, purification, and/or detection, and fusion to an enzyme, fluorescent protein, or luminescent protein, which can provide a marker function.

While the peptides and proteins of the current invention can be chemically synthesized (see, e.g.,“Proteins: Structures and Molecular Principles” (Creighton, ed., W. H. Freeman & Co., N.Y., 1983)), large polypeptides or proteins derived from any of SEQ ID NOS:10-5,504 may advantageously be produced by techniques well-known in the art for expressing genes and/or coding sequences. These techniques include, but are not limited to, in vitro recombinant DNA techniques, synthetic techniques, and in vivo genetic recombination (see, e.g., the techniques described in “Molecular Cloning, A Laboratory Manual”, supra, and “Current Protocols in Molecular Biology”, supra). Alternatively, DNA and/or RNA encoding any of the proteins, polypeptides, or peptides of the present invention may be chemically synthesized using, for example, synthesizers (see, e.g., the techniques described in “Oligonucleotide Synthesis” (Gait, ed., IRL Press, Oxford, 1984)).

A variety of host-expression vector systems may be utilized to express the instant nucleotide sequences. Where the gene product to be produced is a soluble derivative, the gene product can be recovered from the culture, i.e., from the host cell if the peptide or protein is not secreted, and from the culture media if the peptide or protein is secreted by the cells. However, such engineered host cells themselves may be used in situations where it is important not only to retain the structural and functional characteristics of the expressed peptide or protein, but to assess biological activity, e.g., in certain drug screening assays.

The expression systems that may be used in the invention include, but are not limited to: microorganisms such as bacteria (e.g., E. coli, B. subtilis) transformed with a recombinant bacteriophage DNA, plasmid DNA, or cosmid DNA expression vector containing one or more nucleotide sequences from any of SEQ ID NOS:10-5,504; yeast (e.g., Saccharomyces, Pichia) transformed with a recombinant yeast expression vector containing one or more nucleotide sequences from any of SEQ ID NOS:10-5,504; insect cell systems infected with a recombinant virus expression vector (e.g., baculovirus) containing one or more nucleotide sequences from any of SEQ ID NOS:10-5,504; plant cell systems either infected with a recombinant virus expression vector (e.g., cauliflower mosaic virus or tobacco mosaic virus), or transformed with a recombinant plasmid expression vector (e.g., Ti plasmid), containing one or more nucleotide sequences from any of SEQ ID NOS:10-5,504; or mammalian cell systems (e.g., COS, CHO, BHK, 293, 3T3, U937) using a recombinant expression construct comprising one or more nucleotide sequences from any of SEQ ID NOS:10-5,504 and a promoter derived either from the genome of mammalian cells (e.g., metallothionein promoter) or from mammalian viruses (e.g., the adenovirus late or vaccinia virus 7.5K promoter).

In bacterial systems, any of a number of expression vectors may be advantageously selected depending upon the use intended for the gene product being expressed. When large quantities of the gene product are to be produced, for example for the generation of pharmaceutical compositions comprising the gene product, or for raising antibodies to the gene product, vectors that direct the expression of high levels of fusion protein products that are readily purified may be desirable. Such vectors include, but are not limited to: the E. coli expression vector pUR278 (Ruther and Muller-Hill, EMBO J. 2:1791-1794, 1983), in which the coding sequence of the polynucleotide to be expressed may be ligated individually into the vector in frame with the lacZ coding region so that a fusion protein is produced; pIN vectors (Inouye and Inouye, Nucleic Acids Res. 13:3101-3109, 1985, and Van Heeke and Schuster, J. Biol. Chem. 264:5503-5509, 1989); PGEX vectors; and the like. pGEX vectors can be used to express foreign polypeptides as fusion proteins with glutathione S-transferase (GST). Such fusion proteins are generally soluble, and can easily be purified from lysed cells by adsorption to glutathione-agarose beads, followed by elution in the presence of free glutathione. The pGEX vectors are designed to include thrombin or factor Xa protease cleavage sites, so that the cloned target gene product can be released from the GST moiety. However, if the resulting fusion protein is insoluble and forms inclusion bodies in the host cell, the inclusion bodies may be purified and the recombinant protein solubilized using techniques well-known to one of skill in the art.

In an exemplary insect system, Autographa californica nuclear polyhedrosis virus (AcNPV) is used as a vector to express one or more GTS sequence (see, e.g., Smith et al., Mol. Cell. Biol. 3:2156-2165, 1983, and U.S. Pat. No. 4,215,051). In one embodiment of the current invention, Sf9 insect cells are infected with a virus vector that directs the expression of a GTS peptide or protein in the insect cells.

In mammalian host cells, a number of viral-based expression systems may be utilized. Specific embodiments (described more fully below) include, but are not limited to, use of a CMV promoter to transiently express any of the cDNA sequences of the current invention in U937 or Cos-7 cells. Alternatively, retroviral vector systems well-known in the art may be used to insert the recombinant expression construct into host cells. Additionally, in certain embodiments vaccinia virus-based expression systems may be employed.

In yeast, a number of vectors containing constitutive or inducible promoters may be used, see, e.g.: “Current Protocols in Molecular Biology”, supra, Ch. 13; Bitter et al., Methods in Enzymol. 153:516-544, 1987, “DNA Cloning”, Vol. II, Ch. 3 (Glover, ed., IRL Press, Washington, D.C., 1986); Bitter, Methods in Enzymol. 152:673-684, 1987, and “The Molecular Biology of the Yeast Saccharomyces”, Vols. I and II, (Strathern et al., eds., Cold Spring Harbor Press, 1982).

In plants, a number of plant expression vectors can be used, and the expression of the coding sequence may be driven by any of a number of promoters. For example: viral promoters such as the 35S and 19S RNA promoters of CaMV (Brisson et al., Nature 310:511-514, 1984), or the coat protein promoter of TMV (Takamatsu et al., EMBO J. 6:307-311, 1987); plant promoters such as that of the small subunit of RUBISCO (Coruzzi et al., EMBO J. 3:1671-1680, 1984, and Broglie et al., Science 224:838-843, 1984); or heat shock promoters, e.g., soybean hsp17.5-E or hsp17.3-B (Gurley et al., Mol. Cell. Biol. 6:559-565, 1986); may be used. These constructs can be introduced into plant cells using Ti or Ri plasmids, plant virus vectors, direct DNA transformation, microinjection, electroporation, and other methods that are well-known to those of skill in the art, see, e.g., Weissbach and Weissbach, in “Methods for Plant Molecular Biology”, Section VIII, pp. 421-463, (Schuler and Zielinsky, eds., Academic Press, N.Y., 1988), and “Plant Molecular Biology”, 2d Ed., Ch. 7-9 (Grierson, ed., Blackie Academic and Professional, London, 1988).

When an adenovirus is used as an expression vector, the nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the gene product of interest in infected hosts. (see, e.g., Logan and Shenk, Proc. Natl. Acad. Sci. USA 81:3655-3659, 1984). Specific initiation signals may also be required for efficient translation of inserted nucleotide sequences of interest. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of a coding sequence of interest is inserted, exogenous translational control signals, including, perhaps, the ATG initiation codon, may be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enchanter elements, transcription terminators, etc. (see, e.g., Nevins, CRC Crit. Rev. Biochem. 19:307-322, 1986).

In addition, a host cell strain may be chosen that modulates the expression of the inserted sequences, or modifies and processes the gene product, in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the desired modification and processing of the foreign protein expressed. To this end, eukaryotic host cells, which possess the cellular machinery for proper processing of the primary transcript, may be used. Such mammalian host cells include, but are not limited to, CHO, VERO, BHK, Hela, COS, MDCK, 293, 3T3, WI38, and U937 cells.

For long-term, high-yield production of recombinant proteins, stable expression is preferred. For example, cell lines that stably express the sequences disclosed herein may be engineered. Rather than using expression vectors that contain viral origins of replication, host cells can be transformed with DNA controlled by appropriate expression control elements (e.g., promoter, enchanter sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the gene product of interest. Such engineered cell lines may be particularly useful in screening and evaluating compounds that affect the endogenous activity of the gene product of interest.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprt⁻ cells, respectively. Also, anti-metabolite resistance can be used for selection with the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

The gene product of interest can also be expressed in a transgenic animal. Animals of any species, including, but not limited to, mice, rats, rabbits, guinea pigs, pigs, micro-pigs, goats, and non-human primates, e.g., baboons, monkeys, and chimpanzees, may be used to generate transgenic animals that express the instant gene product of interest.

Any technique known in the art may be used to introduce the transgene of interest into animals to produce the founder lines of transgenic animals. Such techniques include, but are not limited to: pronuclear microinjection (U.S. Pat. No. 4,873,191); retrovirus-mediated gene transfer into germ lines (Van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); sperm-mediated gene transfer (Lavitrano et al., Cell 57:717-723, 1989); and positive-negative selection, as described in U.S. Pat. No. 5,464,764. For a review of such techniques, see, e.g., Gordon, Intl. Rev. Cytol. 115:171-229, 1989.

The present invention provides for transgenic animals that carry the transgene of interest in all their cells, as well as animals that carry the transgene in some, but not all, of their cells, i.e., mosaic animals. The transgene may be integrated as a single transgene or in concatamers, e.g., head-to-head tandems or head-to-tail tandems. The transgene may also be selectively introduced into, and activated in, a particular cell-type by following, for example, the teaching of Lasko et al., Proc. Natl. Acad. Sci. USA 89:6232-6236, 1992). The regulatory sequences required for such a cell-type specific activation will depend upon the particular cell-type of interest, and are well-known to those of skill in the art.

When it is desired that the transgene of interest be integrated into the chromosomal site of the endogenous copy of the gene corresponding to the transgene (“the endogenous gene”), gene targeting is preferred. Briefly, in this technique vectors containing some nucleotide sequences homologous to the endogenous gene are designed for the purpose of integrating, via homologous recombination with chromosomal sequences, into and disrupting the function of the endogenous gene. In this way, the expression of the endogenous gene may also be eliminated by inserting non-functional sequences into the endogenous gene. The transgene may also be selectively introduced into a particular cell-type, thus inactivating the endogenous gene in only that cell-type, by following, for example, the teaching of Gu et al., Science 265:103-106, 1994. The regulatory sequences required for cell-type specific inactivation will depend upon the particular cell-type of interest, and will be apparent to those of skill in the art.

Once transgenic animals have been generated, the expression of the recombinant gene of interest may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include, but are not limited to, Northern blot analysis of cell-type samples obtained from the animal, in situ hybridization analysis, and RT-PCR. Samples of gene-expressing tissue may also be evaluated immunocytochemically using antibodies selective for the transgene product, as described below.

### 6.3 Cells that Contain a Disrupted Allele of a Gene Encoding a Polynucleotide of the Current Invention

Another aspect of the current invention are cells that contain a disrupted version of a gene that corresponds to a sequence of the current invention. There are a variety of techniques that can be used to disrupt a gene in a cell, and especially an ES cell. Examples of such methods are described in co-pending U.S. patent application Ser. No. 08/728,963, and U.S. Pat. Nos. 5,789,215, 5,487,992, 5,627,059, 5,631,153, 6,087,555, 6,136,566, 6,139,833, and 6,207,371.

### 6.3.1 Identification of Cells that Express Genes Encoding Polynucleotides of the Current Invention

Host cells that contain a coding sequence and/or express a biologically active gene product of the present invention, or fragment thereof, may be identified by at least four general approaches; (a) DNA-DNA or DNA-RNA hybridization; (b) the presence or absence of “marker” gene functions; (c) assessing the level of transcription of the coding sequence as measured by the expression of mRNA transcripts in the host cell; and (d) detection of a gene product of the present invention, as measured by immunoassay, enzymatic assay, chemical assay, or the biological activity of the gene product. Prior to screening for gene expression, the host cells can first be treated in an effort to increase the level of expression of sequences encoding the gene products of the present invention, especially in cell lines that produce low amounts of mRNAs and/or peptides and proteins.

In approach (a) above, the presence of a coding sequence of the present invention can be detected by DNA-DNA or DNA-RNA hybridization using probes comprising nucleotide sequences that are homologous or complementary to a coding sequence of the present invention, or portions or derivatives thereof.

In approach (b), the recombinant expression vector/host system can be identified and selected based upon the presence or absence of certain “marker” gene functions (e.g., thymidine kinase activity, resistance to antibiotics, resistance to methotrexate, transformation phenotype, occlusion body formation in baculovirus, etc.). For example, if a coding sequence of the present invention is inserted within a marker gene sequence of a vector, recombinants containing the coding sequence can be identified by the absence of the marker gene function. Alternatively, a marker gene can be placed in tandem with a coding sequence of the present invention, under the control of the same or a different promoter used to control the expression of the coding sequence. Expression of the marker gene product in response to induction or selection indicates the presence of the coding sequence.

In approach (c), transcriptional activity of a coding region of the present invention can be assessed by hybridization assays. For example, RNA can be isolated and analyzed by Northern blot using a probe derived from a coding region of the present invention, or any portion thereof. Alternatively, total nucleic acids of the host cell may be extracted and assayed for hybridization to such probes. Additionally, RT-PCR (using oligonucleotides from a coding region of the present invention) may be used to detect low levels of gene expression in a sample, in RNA isolated from a spectrum of different tissues, or in cDNA libraries derived from different tissues, to determine which tissues express a given coding region of the present invention.

In approach (d), the expression of the peptides and proteins of the present invention can be assessed immunologically, for example by Western blots, immunoassays such as radioimmuno-precipitation, enzyme-linked immunoassays, and the like. This can be achieved by using an antibody, or a binding partner, that selectively binds to a peptide or protein of the present invention, as described below.

### 6.4 Antibodies to Gene Products of the Current Invention

Antibodies that selectively or specifically recognize one or more epitopes of a peptide or protein of the current invention, or epitopes of conserved variants of a peptide or protein at least partially encoded by a coding region of the present invention, or any and all peptide fragments thereof, are also encompassed by the invention. Such antibodies include, but are not limited to, polyclonal, monoclonal, humanized, chimeric, and single chain antibodies, Fab and F(ab′)₂ fragments, fragments produced by a Fab expression library, anti-idiotypic (anti-Id) antibodies, and epitope-binding fragments of any of the above.

The antibodies of the invention may be used, for example, in the detection of a gene product of interest of the current invention in a biological sample and can thus be utilized as part of diagnostic or prognostic techniques where patients may be tested for abnormal amounts of these peptides or proteins. Such antibodies may also be utilized in conjunction with, for example, compound screening schemes, as described below, for the evaluation of the effect of test compounds on expression and/or activity of the gene products of the current invention of interest. Additionally, such antibodies can be used in conjunction with gene therapy and gene delivery techniques, as described herein, to evaluate normal and/or engineered cells that express a gene product of the present invention prior to introduction into a patient, or as a part of treatment methods for development and/or cell differentiation disorders. Such antibodies can also be used to inhibit the activity of a peptide or protein of the present invention.

To produce antibodies, a suitable host animal may be immunized by injection with a peptide or protein of the present invention, a subunit, truncation, functional equivalent, or mutant of such a peptide or protein, or denatured forms of the above. Suitable host animals include, but are not limited to, rabbits, mice, and rats, to name but a few. Depending on the host species, various adjuvants may be used to increase the immunological response, including, but not limited to, Freund's adjuvant (complete and incomplete), mineral salts such as aluminum hydroxide or phosphate, surface active substances such as lysolecithin, pluronic polyols, polyanions, peptides, oil emulsions, and potentially useful human adjuvants such as BCG (Bacille Calmette-Guerin) and Corynebacterium parvum. The immune response can also be enhanced by combination and/or coupling with molecules such as keyhole limpet hemocyanin, ovalbumin, tetanus or diphtheria toxoid, cholera toxin, or fragments thereof. Polyclonal antibodies are heterogeneous populations of antibody molecules derived from the sera of the immunized animals.

Monoclonal antibodies (MAbs), which are homogeneous populations of antibodies to a particular antigen, may be obtained by any technique that provides for the production of antibody molecules by continuous cell lines in culture. These include, but are not limited to, the hybridoma technique (Kohler and Milstein, Nature 256:495-497, 1975, and U.S. Pat. No. 4,376,110), the human B-cell hybridoma technique (Kosbor et al., Immunology Today 4:72, 1983, and Cole et al., Proc. Natl. Acad. Sci. USA 80:2026-2030, 1983), and the EBV-hybridoma technique (Cole et al., in “Monoclonal Antibodies and Cancer Therapy”, Vol. 27, UCLA Symposia on Molecular and Cellular Biology, New Series, pp. 77-96 (Reisfeld and Sell, eds., Alan R. Liss, Inc. N.Y., 1985)). Such antibodies may be of any immunoglobulin class, including IgG, IgM, IgE, IgA, and IgD, and any subclass thereof. A hybridoma producing a mAb of the invention may be cultivated in vitro or in vivo. In certain instances, production of high titers of mAbs in vivo is the preferred method of production.

In addition, techniques developed for the production of “chimeric antibodies” (Morrison et al., Proc. Natl. Acad. Sci. USA 81:6851-6855, 1984, Neuberger et al., Nature 312:604-608, 1984, and Takeda et al., Nature 314:452-454, 1985) can be used, for example by splicing the genes from a mouse antibody molecule of appropriate antigen specificity together with genes from a human antibody molecule of appropriate biological activity. A chimeric antibody is one in which different portions are derived from different animal species, such as those having a variable region derived from a porcine mAb, and a human immunoglobulin constant region. Such technologies are described in U.S. Pat. Nos. 6,075,181 and 5,877,397.

Alternatively, techniques described for the production of single chain antibodies (U.S. Pat. No. 4,946,778, Bird, Science 242:423-426, 1988, Huston et al., Proc. Natl. Acad. Sci. USA 85:5879-5883, 1988, and Ward et al., Nature 341:544-546, 1989) can be adapted to produce single chain antibodies against a gene product of the present invention of interest. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: F(ab′)₂ fragments, which can be produced by pepsin digestion of an antibody molecule; and Fab fragments, which can be generated by reducing the disulfide bridges of F(ab′)₂ fragments. Alternatively, Fab expression libraries may be constructed (Huse et al., Science 246:1275-1281, 1989) to allow rapid and easy identification of monoclonal Fab fragments with the desired specificity.

Antibodies to the peptides and proteins of the present invention, or fragments or truncated versions thereof, can in turn be utilized to generate anti-idiotypic antibodies that “mimic” an epitope of the peptide or protein, using techniques well-known to those skilled in the art (see, e.g., Greenspan and Bona, FASEB J. 7:437-444, 1993, and Nissinoff, J. Immunol. 147:2429-2438, 1991). For example, antibodies that bind to a peptide or protein of the current invention and competitively inhibit the binding of such a peptide or protein to any of its binding partners in the cell, can be used to generate anti-idiotypes that “mimic” the peptide or protein and, therefore, bind and neutralize a particular binding partner of the peptide or protein. Such neutralizing antibodies, anti-idiotypes, Fab fragments of such antibodies, or humanized derivatives thereof, can be used in therapeutic regimens to mimic or neutralize (depending on the antibody) the effect of a particular peptide or protein, or a particular binding partner of a peptide or protein, of interest.

### 6.5 Diagnosis of Disorder Affecting Development and Cell Differentiation

A variety of methods can be employed for the diagnostic and prognostic evaluation of disorders involving developmental and/or differentiation processes, and for identification of subjects having a predisposition to such disorders, which may, for example, utilize reagents such as the instant nucleotide sequences and/or antibodies to the instant gene products. For example, such reagents may be used to detect: (1) mutations within, or over- or under-expression of, the respective mRNAs relative to the non-disorder state; (2) an over- or under-abundance of the respective gene product relative to the non-disorder state; and (3) perturbations or abnormalities in the intra- and/or inter-cellular processes mediated by the peptides or proteins of the current invention.

The methods described herein may be performed, for example, by utilizing pre-packaged diagnostic kits comprising at least one specific nucleotide sequence or antibody reagent of the present invention, which may be conveniently used, e.g., in clinical settings, to diagnose patients exhibiting developmental or cell differentiation disorder abnormalities.

For the detection of mutations in any of the genes of the present invention, any nucleated cell can be used as a starting source for genomic nucleic acid. For the detection of gene expression or gene products, any cell-type or tissue in which the gene of interest is expressed, such as, for example, ES cells, may be utilized. Examples of cells and/or tissues that can be analyzed using the polynucleotides and/or antibodies of the present invention include, but are not limited to, endothelial cells, epithelial cells, islets, neurons or neural tissue, mesothelial cells, osteocytes, lymphocytes, chondrocytes, hematopoietic cells, immune cells, cells of the major glands or organs (e.g., lung, heart, stomach, pancreas, kidney, skin, etc.), exocrine and/or endocrine cells, embryonic and other stem cells, fibroblasts, and culture adapted and/or transformed versions of the above.

In addition to developmental and/or cell differentiation disorders or abnormalities, diseases or natural processes that can be correlated with the expression of the disclosed GTSs (mutant or normal) include, but are not limited to, aging, cancer, autoimmune disease, lupus, scleroderma, inflammatory bowel disease, Crohn's disease, multiple sclerosis, glandular disorders, immune disorders, schizophrenia, inflammatory disorders, psychosis, alopecia, ataxia telangiectasia, osteo and rheumatoid arthritis, diabetes, skin disorders such as acne, eczema, and the like, high blood pressure, pulmonary disease, atherosclerosis, cardiovascular disease, degenerative diseases of the neural or skeletal systems, Alzheimer's disease, Parkinson's disease, osteoporosis, asthma, genetic birth defects, infertility, epithelial ulcerations, and viral, parasitic, fungal, yeast, or bacterial infections.

Primary, secondary, or culture-adapted variants of cancer cells/tissues can also be analyzed using the polynucleotides and/or antibodies, or fragments thereof, of the present invention. Examples of such cancers include, but are not limited to: Cardiac: sarcoma (angiosarcoma, rhabdomyosarcoma, fibrosarcoma, liposarcoma), myxoma, fibroma, lipoma, teratoma and rhabdomyoma; Lung: bronchogenic carcinoma (squamous cell, undifferentiated small or large cell, adenocarcinoma), alveola (bronchiola) carcinoma, bronchial adenoma, sarcoma, lymphoma, chondromatous hamartoma, mesothelioma; Gastrointestinal: esophagus (adenocarcinoma, leiomyosarcoma, squamous cell carcinoma, lymphoma), stomach (carcinoma, leiomyosarcoma, lymphoma), pancreas (gastrinoma, carcinoid tumors, vipoma, ductal adenocarcinoma, insulinoma, glucagonoma), small bowel (adenocarcinoma, lymphoma, carcinoid tumors, neurofibroma, Karposi's sarcoma, leiomyoma, hemangioma, lipoma, fibroma), large bowel (adenocarcinoma, tubular adenoma, villous adenoma, hamartoma, leiomyoma); Genitourinary tract: kidney (Wilm's tumor [nephroblastoma], adenocarcinoma, lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), hepatoblastoma, hemangioma, angiosarcoma, cholangiocarcinoma, hepatocellular adenoma; Bone: osteogenic sarcoma (osteosarcoma), malignant fibrous histiocytpma, fibrosarcoma, chondrosarcoma, malignant lymphoma (reticulum cell sarcoma), multiple myeloma, malignant giant cell tumor, chordoma, Ewing's sarcoma, osteochronfroma (osteocartilaginous exostoses), benign chondroma, osteoid osteoma, chondroblastoma, chondromyxofibroma, and giant cell tumors; Nervous system: skull (osteoma, hemangioma, granuloma, xanthoma, osteitis deformans), meninges (meningiosarcoma, meningioma, gliomatosis), brain (astrocytoma, medulloblastoma, glioma, ependymoma, germinoma [pinealoma], glioblastoma multiforme, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovary (ovarian carcinoma [clear cell carcinoma, serous cystadenocarcinoma, mucinous cystadenocarcinoma, endometrioid tumors, celioblastoma, unclassified carcinoma], granulosa-thecal cell tumors, Sertoli-Leydig cell tumors, dysgerminoma, malignant teratoma), vulva (intraepithelial carcinoma, melanoma, squamous cell carcinoma, adenocarcinoma, fibrosarcoma), vagina (clear cell carcinoma, squamous cell carcinoma, botryoid sarcoma [embryonal rhabdomyosarcoma]), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, squamous cell carcinoma, basal cell carcinoma, Karposi's sarcoma, dysplastic nevi, moles, lipoma, angioma, dermatofibroma, keloids, psoriasis; Breast: carcinoma and sarcoma; and Adrenal glands: neuroblastoma.

Detection techniques that can be used to conduct the above analyses are described in greater detail below.

### 6.5.1. Detection of Genes of the Current Invention and Their Respective Transcripts

Mutations within the genes of the current invention can be detected utilizing a number of techniques. Nucleic acid from any nucleated cell can be isolated according to standard procedures that are well-known to those of skill in the art, and used as the starting point for such assay techniques.

DNA may be used in hybridization and/or amplification assays of biological samples to detect abnormalities involving gene structure, including chromosomal rearrangements, point mutations, insertions and deletions. Such assays include, but are not limited to, Southern, single stranded conformational polymorphism (SSCP), and PCR analyses. Also, well-known genotyping techniques can be performed to identify an individual carrying a mutation in any of the instant genes. Such techniques include, for example, the use of restriction fragment length polymorphisms (RFLPs), which involve sequence variations in one or more of the recognition sites for a particular restriction enzyme used in the analysis.

Such diagnostic methods for the detection of such gene structure abnormalities can involve, for example, contacting and incubating nucleic acids obtained from a sample, e.g., derived from a patient sample and/or other appropriate cellular source, with one or more labeled nucleic acid reagents, under conditions favorable for specific annealing of these reagents to their complementary sequences within the sample nucleic acids. Examples of nucleic acids that can be obtained from a sample and/or used as labeled nucleic acid reagents include, but are not limited to, recombinant DNA molecules, cloned genes, or degenerate variants thereof. Generally, the labeled nucleic acid reagents are at least 15 to 30 nucleotides in length. After incubation, the non-annealed nucleic acids are removed from the nucleic acid molecule hybrid. The presence of nucleic acids that have hybridized, if any such molecules exist, is then detected.

In such detection schemes, the nucleic acids from the cell-type and/or tissue of interest can be immobilized, for example, to a solid support such as a membrane, or a plastic surface such as that on a microtiter plate or polystyrene beads. After incubation in such schemes, the non-annealed labeled nucleic acid reagents are easily removed. Detection of the remaining annealed labeled nucleic acid reagents can be accomplished using standard techniques well-known to those in the art. The annealing pattern obtained can be compared to the annealing pattern expected from a normal gene sequence, in order to determine whether a gene mutation is present.

Alternative diagnostic methods for detection of gene-specific nucleic acid molecules, in patient samples and/or other appropriate cell sources, may involve amplification, e.g., by PCR (see, e.g., U.S. Pat. No. 4,683,202), followed by detection of the amplified molecules using techniques well-known to those of skill in the art. The resulting amplified sequences can be compared to those expected if the nucleic acid being amplified contained only normal copies of the respective gene, to determine whether a gene mutation exists.

Furthermore, the polynucleotide sequences of the current invention may be mapped to chromosomes, and specific regions of chromosomes, using well-known genetic and/or chromosomal mapping techniques. These techniques include in situ hybridization, linkage analysis against known chromosomal markers, hybridization screening with libraries or flow-sorted chromosomal preparations specific to known chromosomes, and the like. The technique of fluorescent in situ hybridization of chromosome spreads has been described, for example, in “Human Chromosomes: A Manual of Basic Techniques” (Verma and Babu, eds., Pergamon, Oxford, 1989). Fluorescent in situ hybridization of chromosomal preparations, as well as other physical chromosome mapping techniques, may be correlated with additional genetic map data. Examples of genetic map data can be found, for example, in “Genetic Maps: Locus Maps of Complex Genomes” Book 5: Human Maps, (O'Brien, ed., Cold Spring Harbor Laboratory Press, Cold Spring Harbor, N.Y., 1990). Comparisons of physical chromosomal map data may be of particular interest in detecting genetic diseases in carrier states.

Additionally, the level of expression of the genes of the present invention can be assayed, by detecting and measuring the transcription of such genes. For example, RNA from a cell-type or tissue known to express, or suspected of expressing, any of the genes of the current invention can be isolated and tested utilizing hybridization and/or PCR techniques (e.g., northern blot and/or RT-PCR). Such analyses may reveal both quantitative and qualitative aspects of the expression pattern of the respective gene, including activation or inactivation of gene expression. Also, in situ hybridization, using a suitable radioactively, enzymatically, or chemically labeled probe based on the instant nucleotide sequences, can be used to assess expression patterns in vivo.

GTS oligonucleotides can also be used as hybridization probes for screening libraries and assessing gene expression patterns (particularly using a microarray or high-throughput “chip” format). Such GTS hybridization probes can also be used in conjunction with a solid support matrix/substrate (e.g., resins, beads, membranes, plastics, polymers, metal or metallized substrates, crystalline or polycrystalline substrates, etc.). Of particular note are spatially addressable arrays (i.e., gene chips, microtiter plates, etc.) of oligonucleotides and/or polynucleotides, or corresponding oligopeptides and/or polypeptides, wherein at least one of the biopolymers present on the spatially addressable array comprises an oligonucleotide or polynucleotide sequence from at least one of SEQ ID NOS:10-5,504, or an amino acid sequence encoded thereby. Methods for attaching biopolymers to, or synthesizing biopolymers on,, solid support matrices, and conducting binding studies thereon, are disclosed in U.S. Pat. Nos. 5,700,637, 5,556,752, 5,744,305, 4,631,211, 5,445,934, 5,252,743, 4,713,326, 5,424,186, and 4,689,405.

Additionally, the presently described GTSs, or primers derived therefrom, can be used to screen spatially addressable arrays, or pools therefrom, of clones present in a full-length human cDNA library. The 96 well microtiter plate format is especially well suited to the screening, by PCR for example, of pooled subfractions of cDNA clones.

Addressable arrays comprising oligonucleotide sequences from any of SEQ ID NOS:10-5,504 can also be used to identify and characterize the temporal and/or tissue specific expression of the corresponding genes. These arrays use oligonucleotide sequences of sufficient length to confer the desired specificity, yet be within the limitations of the production technology. The probes are generally between from about 8 to about 2000 nucleotides in length. In certain embodiments, the probes consist of 60 contiguous, or 25 contiguous, nucleotides from any of SEQ ID NOS:10-5,504.

For example, a series of GTS oligonucleotide sequences, or the complements thereof, can be used in chip format to represent all, or a portion, of a particular GTS sequence. The oligonucleotides are typically between about 16 to about 40 nucleotides in length (or any whole number within the stated range). The GTS sequences can be represented using oligonucleotides that partially overlap each other, and/or using oligonucleotides that do not overlap. Accordingly, a particular GTS sequence can be represented on a chip by at least two or three distinct oligonucleotides that comprise at least 8 contiguous nucleotides from the particular GTS sequence, or the complement thereof. Such oligonucleotides can begin at any nucleotide position within a GTS sequence, and continue in either a sense (5′-to-3′) or antisense (3′-to-5′) orientation vis-a-vis the GTS sequence.

Microarray-based analysis allows the discovery of broad patterns of genetic activity, providing new understanding of gene functions, and generating novel and unexpected insight into transcriptional processes and biological mechanisms. The use of addressable arrays comprising sequences first disclosed in SEQ ID NOS:10-5,504 provides detailed information about transcriptional changes involved in a specific pathway, potentially leading to identification of novel components or gene functions that manifest themselves as novel phenotypes.

Probes consisting of sequences first disclosed in SEQ ID NOS:10-5,504 can also be used in the identification, selection, and validation of novel molecular targets for drug discovery. The use of these unique sequences permits the direct confirmation of drug targets, and recognition of drug-dependent changes in gene expression that are modulated through pathways distinct from the intended target of the drug. These unique sequences therefore also have utility in defining and monitoring both drug action and toxicity.

As just one example of utility, any one or more of the sequences first disclosed in SEQ ID NOS:10-5,504 can be utilized in microarrays, or other assay formats, to screen collections of genetic material from patients who have a particular medical condition. These investigations can also be carried out in silico, and by comparing previously collected genetic databases with the disclosed GTS sequences using computer software known to those in the art.

Although the disclosed GTSs have been specifically described using nucleotide sequence, it should be appreciated that each of the GTSs can uniquely be described using any of a wide variety of additional structural attributes, or combinations thereof. For example, a given GTS can be described by the net composition of the nucleotides present within a given region of the GTS, in conjunction with the presence of one or more specific oligonucleotide sequence(s) first disclosed in the GTS. Alternatively, a restriction map specifying the relative positions of restriction endonuclease digestion sites, or various palindromic or other specific oligonucleotide sequences, can be used to structurally describe a given GTS. Such restriction maps, which are typically generated by widely available computer programs (e.g., the University of Wisconsin GCG sequence analysis package (SEQUENCHER 3.0, Gene Codes Corp.), etc.), can also be used in conjunction with one or more discrete nucleotide sequence(s) present in a GTS, which can be described by the position of the sequence relative to one or more additional sequence(s) or restriction sites in the GTS.

### 6.5.2 Detection of Gene Products of the Current Invention

Antibodies directed against wild-type or mutant gene products of the current invention, or conserved variants or peptide fragments thereof, which are discussed above in Section 6.4, may be used as diagnostics and/or prognostics for disorders affecting development and cellular differentiation. Such diagnostic and prognostic methods may be used to detect abnormal levels of gene expression, and/or abnormal structure or location (temporal, tissue, cellular, or subcellular) of the respective gene product. Such assays may be performed in vivo or in vitro, for example on biopsy tissue.

The tissue or cell-type to be analyzed generally include those that are known to contain, or suspected of containing, cells that express the respective gene. The protein isolation methods employed herein may, for example, be those previously described (“Antibodies: A Laboratory Manual”, supra). The isolated cells can be derived from cell culture or a patient. The analysis of cells taken from culture may be a necessary step in assessment of cells that can be used as part of a cell-based gene therapy technique, or, alternatively, to test the effect of compounds on expression of the respective gene.

The presently described antibodies, antibody fragments, and fusion proteins are also useful to quantitatively or qualitatively detect the presence of gene products of the current invention, or conserved variants or peptide fragments thereof. This can be accomplished, for example, by immunofluorescence techniques employing a fluorescently labeled antibody, antibody fragment, or fusion protein, with light microscopic, flow cytometric, or fluorimetric detection.

The antibodies, antibody fragments, and/or fusion or conjugated gene products of the present invention may also be employed histologically, for example in immunofluorescence, immunoelectron microscopy, in situ detection, non-immuno assays, and/or for catalytic subunit binding analysis. The binding activity of each lot of antibody, antibody fragment, or fusion protein can be determined according to well-known methods, which can be used to determine operative and optimal assay conditions by employing routine experimentation.

In situ detection may be accomplished by removing a histological specimen from a patient, and applying thereto a labeled antibody, a fragment thereof, or fusion protein of the present invention, preferably by overlaying onto a biological sample. Through the use of such a procedure, it is possible to determine the presence and distribution of a gene product, conserved variant, or peptide fragment of the current invention in the examined tissue. Those of ordinary skill will readily perceive that any of a wide variety of histological methods (such as staining procedures) can be modified in order to achieve such in situ detection.

Immunoassays and non-immunoassays for any of the gene products of the current invention will typically comprise incubating a biological sample, such as a biological fluid, a tissue extract, freshly harvested cells, or lysates of cells that have been incubated in cell culture, in the presence of a labeled antibody, antibody fragment, or fusion protein able to selectively bind to the respective gene product of interest, and detecting the bound antibody, fragment, or fusion protein by any of a number of techniques well-known in the art.

The biological sample may be brought in contact with and immobilized onto a solid phase support or carrier, such as nitrocellulose, that is capable of immobilizing cells, cell particles or soluble proteins. The support may then be washed with one or more suitable buffers, followed by treatment with a labeled antibody, antibody fragment, or fusion protein that selectively binds to the particular gene product of interest. The solid phase support may then be washed with buffer(s) to remove unbound label. The amount of label remaining on the solid support may then be detected by conventional means.

The term “solid phase support or carrier” encompasses any support capable of binding an antigen, antibody, cell, or cell particle. Well-known supports include glass, polypropylene, polyethylene, dextran, nylon, amylases, natural and modified celluloses, polyacrylamides, gabbros, and magnetite. The nature of the carrier can be soluble to some extent, or insoluble, for the purposes of the present invention. The support material may have virtually any possible structural configuration, as long as the bound moiety is capable of binding to an antigen, antibody, antibody fragment, or fusion protein. Thus, the support configuration may be spherical, as a bead, cylindrical, as the inside surface of a test tube or the external surface of a rod, or flat, as a sheet or test strip. Preferred supports include, but are not limited to, polystyrene beads, with others known to skilled artisans, or determined with routine experimentation.

An antibody or fragment thereof can be detectably labeled by linkage to an enzyme, and used in an enzyme immunoassay (see, e.g., Voller, Diagnostic Horizons 2:1-7, 1978 (Microbiological Associates Quarterly Publication, Walkersville, Md.), Voller et al., J. Clin. Pathol. 31:507-520, 1978, Butler, Meth. Enzymol. 73:482-523, 1981, “Enzyme Immunoassay” (Maggio, ed., CRC Press, Boca Raton, Fla., 1980), “Enzyme Immunoassay” (Ishikawa et al., eds., Kgaku-Shoin, Tokyo, Japan, 1981)). The enzyme bound to the antibody or antibody fragment can react with an appropriate substrate, for example a chromogenic substrate, to produce a chemical moiety that can be detected, for example, by visual, fluorimetric, or spectrophotometric means. Enzymes that can be used in such techniques include, but are not limited to, staphylococcal nuclease, malate dehydrogenase, delta-5-steroid isomerase, yeast alcohol dehydrogenase, horseradish peroxidase, urease, triose phosphate isomerase, alkaline phosphatase, catalase, ribonuclease, asparaginase, glucose oxidase, glucoamylase, beta-galactosidase, alpha-glycerophosphate dehydrogenase, glucose-6-phosphate dehydrogenase, and acetylcholinesterase.

Alternatively, radioactive labeling of the antibody or antibody fragment allows detection of the peptide or protein of interest through the use of a radioimmunoassay. The radioactivity can be detected, for example, using a gamma counter, a scintillation counter, or by autoradiography.

It is also possible to label an antibody or antibody fragment with a fluorescent compound. When the fluorescent label is exposed to light of the proper wavelength, its presence can be detected due to fluorescence. Among the most commonly used fluorescent labeling compounds are fluorescein isothiocyanate, rhodamine, phycoerythrin, phycocyanin, allophycocyanin, and fluorescamine. An antibody or antibody fragment can also be labeled using fluorescence emitting metals such as ¹⁵²Eu, or others of the lanthanide series, which can be attached to an antibody or antibody fragment using such metal chelating groups as diethylenetriaminepentacetic acid (DTPA) or ethylenediaminetetraacetic acid (EDTA).

An antibody or antibody fragment also can be detectably labeled by coupling to a chemiluminescent compound, such as luminol, isoluminol, theromatic acridinium ester, imidazole, acridinium salt, or oxalate ester. The labeled antibody or antibody fragment can be detected by luminescence that arises during the course of a chemical reaction. Additionally, a bioluminescent compound, such as luciferin, luciferase, or aequorin, can be used to label an antibody or antibody fragment. Bioluminescence is a type of chemiluminescence found in biological systems, in which a catalytic protein increases the efficiency of the chemiluminescent reaction. The presence of a bioluminescent protein is once again determined by detecting the presence of luminescence.

### 6.6 Screening Assays for Compounds that Modulate the Expression or Activity of the Instant Gene Products

A number of assays can be used to identify compounds that modulate the expression and/or activity of peptides and/or proteins at least partially encoded by any one or more of SEQ ID NOS:10-5,504 (i.e., the instant gene products). Such compounds may be useful, for example, in elaborating the biological function of the instant gene products, and for ameliorating disorders affecting development and/or cell differentiation. Such assays can be used to identify: compounds that interact with (e.g., bind to) the instant gene products; compounds that interact with intracellular proteins that interact with the instant gene products; compounds that interfere with or enhance the interaction of the instant gene products, either with each other, or with other intracellular proteins that interact with the instant gene products; and compounds that modulate the activity of the instant gene products (i.e., modulate the splicing or level of expression of nucleic acids that encode the instant gene products, or the level of the instant gene products themselves; see, e.g., Platt et al., J. Biol. Chem. 269:28558-28562, 1994).

Compounds that can be screened in such assays include, but are not limited to, proteins, peptides, antibodies, antibody fragments, prostaglandins, lipids, and other organic (e.g., terpines and peptidomimetics) or inorganic compounds. Such compounds can mimic the activity triggered by the natural ligand (i.e., agonists), or inhibit the activity triggered by the natural ligand (i.e., antagonists), including compounds that bind to and “neutralize” the natural ligand. Such compounds include those that can, for example, affect cell differentiation and/or development by: gaining entry into a particular cell (e.g., an ES cell) and affecting expression of a gene encoding one or more of the instant gene products or another gene involved in development and cell differentiation (e.g., by interacting with the regulatory region directly or with transcription factors involved in gene expression; and/or modulating the activity of one or more of the instant gene products (e.g., by inhibiting or enhancing binding to another cellular peptide, protein, or other factor, involved in function, catalysis, signal transduction, etc.).

Such compounds include, but are not limited to: peptides, including soluble peptides, from random peptide libraries (see, e.g., Lam et al., Nature 354:82-84, 1991, and Houghten et al., Nature 354:84-86, 1991), combinatorial chemistry-derived peptide libraries made from D- and/or L-configuration amino acids, phosphopeptides (such as members of random or partially degenerate directed phosphopeptide libraries; see, e.g., Songyang et al., Cell 72:767-778, 1993); antibodies (including, but not limited to, polyclonal, monoclonal, humanized, anti-idiotypic, chimeric, and single chain antibodies); and antibody fragments (including, but not limited to, Fab, F(ab′)₂ and Fab expression library fragments, and epitope-binding fragments thereof).

Computer modeling and searching technologies permit the identification of compounds, or the improvement of already identified compounds, that can modulate the expression or activity of the instant gene products. Having identified such compound or composition, the active sites or regions can be identified. Such active sites might typically be the binding partner sites, such as, for example, the interaction domains of the instant gene products with their respective binding partners. The active site can be identified using methods known in the art including, for example, comparison to the amino acid sequences, or coding sequences, of active sites of known proteins, or from study of complexes of a particular compound with its natural ligand, for example using chemical or X-ray crystallographic methods to identify the region of the ligand to which the compound binds.

The three dimensional geometric structure of the active site can then be determined using standard methods, including X-ray crystallography, which can determine a complete molecular structure, and solid or liquid phase NMR, which can be used to determine certain intra-molecular distances. Other experimental methods of structure determination can also be used to obtain a partial or complete geometric structure. The geometric structure may be measured with a complexed ligand, either natural or artificial, which may increase the accuracy of the determined active site structure.

If an incomplete or insufficiently accurate structure is determined, computer based numerical modeling methods can be used to complete the structure or improve the accuracy. A number of modeling methods are known to the skilled artisan, including, but not limited to, parameterized models specific to particular biopolymers such as proteins or nucleic acids, molecular dynamics models based on computing molecular motions, statistical mechanics models based on thermal ensembles, and combined models. For most types of models, standard molecular force fields, representing the forces between constituent atoms and groups, are necessary, and can be selected from force fields known in physical chemistry. The incomplete or less accurate structure can serve as a constraint on the structure computed by these methods.

Based on the structure of the active site, determined experimentally, by modeling, or a combination thereof, candidate modulating compounds can be identified by searching databases containing compounds and information on their molecular structure. Such a search seeks compounds having structures that match the determined active site structure and that interact with the groups defining the active site. Such a search can be manual, but is preferably computer assisted.

Additionally, these methods can be used to identify improved modulating compounds based on known modulating compounds or ligands. The composition of the known compound can be modified, and the structural effect of the modification determined, using the experimental and computer modeling methods described above. The altered structure is then compared to the active site structure to determine if an improved fit or interaction results. In this manner, systematic variations, such as by varying side groups, can be quickly evaluated to obtain modified modulating compounds or ligands with improved specificity and/or activity.

Further examples of molecular modeling systems are the CHARM and QUANTA programs (Polygon Corporation, Waltham, Mass.). CHARM performs the energy minimization and molecular dynamics functions. QUANTA performs the construction, graphic modeling and analysis of molecular structure, and allows interactive construction, modification, visualization, and analysis of the behavior of molecules with each other.

A number of articles review computer modeling of drugs interactive with specific proteins, such as Rotivinen et al., Acta Pharmaceutical Fennica 97:159-166, 1988, Ripka, New Scientist, pp. 54-57, Jun. 16, 1988, McKinaly and Rossmann, Ann. Rev. Pharmacol. Toxiciol. 29:111-122, 1989, Perry and Davies, in “QSAR: Quantitative Structure-Activity Relationships in Drug Design”, pp. 189-193 (Fauchere, ed., Alan R. Liss, Inc., N.Y., 1989), Lewis and Dean, Proc. R. Soc. Lond. 236:125-140 and 141-162, 1989, and, with respect to a model receptor for nucleic acid components, Askew et al., J. Am. Chem. Soc. 111:1082-1090, 1989. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario, Canada). Although primarily designed for application to drugs specific to particular proteins, these can be adapted to the design of drugs specific to regions of DNA or RNA, once that region is identified.

Although described above with reference to design and generation of compounds that could alter binding, libraries of known compounds, including natural products or synthetic chemicals, and biologically active materials, including proteins, can also be screened for modulating compounds.

### 6.6.1 In Vitro Screening Assays for Compounds that Bind to the Peptides and Proteins of the Current invention

A number of assays are available to identify compounds that bind to the instant gene products. In general, these assays involve preparing a reaction mixture of one or more of the instant gene products and a test compound under conditions and for a time sufficient to allow the two components to interact and bind, thus forming a complex that can be removed from and/or detected in the reaction mixture. The particular form of the instant gene products used can vary depending upon the goal of the screening assay. For example, where agonists of the natural ligand are sought, the full length gene product, a subunit of the gene product that binds the natural ligand, or a fusion protein containing either of these gene products fused to a protein or polypeptide that affords advantages in the assay system (e.g., labeling, isolation of the resulting complex, etc.) can be utilized.

These screening assays can be conducted various ways. One method of such an assay involves anchoring the gene product of interest, a fusion protein thereof, or the test compound, onto a solid phase and detecting gene product/test compound complexes anchored on the solid phase at the end of the reaction. In one such method, the gene product of interest may be anchored onto a solid surface, and the test compound, which is not anchored, may be labeled, either directly or indirectly. In another embodiment, the gene product of interest anchored on the solid phase is complexed with its natural ligand, and a test compound assayed for the ability to disrupt the gene product/natural ligand complex.

Microtiter plates may conveniently be utilized as the solid phase. The anchored component may be immobilized by non-covalent or covalent attachments. Non-covalent attachment may be accomplished by simply coating the solid surface with a solution of the gene product and drying. Alternatively, an immobilized antibody, preferably a monoclonal antibody, specific for the gene product to be immobilized may be used to anchor the gene product to the solid surface. The surfaces may be prepared in advance and stored.

To conduct such an assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. When the nonimmobilized component is pre-labeled, the detection of label immobilized on the surface indicates that complexes were formed. When the nonimmobilized component is not pre-labeled, an indirect label can be used to detect complexes anchored on the surface, e.g., using a labeled antibody specific for the previously nonimmobilized component (the antibody may be directly labeled or indirectly labeled with a labeled anti-Ig antibody).

Alternatively, a reaction can be conducted in a liquid phase. After the reaction, unreacted components are separated from any complexes formed. Complexes can be detected, e.g., using an immobilized antibody specific for one component of the reaction, for example the gene product or test compound, to anchor complexes formed in solution, and a labeled antibody specific for the other component to detect anchored complexes.

Additionally, a phage display (or other peptide library/binding) system can be used to screen for compounds that bind to one or more of the instant gene products (see, e.g., U.S. Pat. Nos. 5,270,170 and 5,432,018), and peptide arrays comprising a gene product of the present invention can be generated and screened, essentially as described in U.S. Pat. Nos. 5,143,854, 5,405,783, and 5,252,743.

### 6.6.2 Assays for Intracellular Proteins that Interact with the Peptides and Proteins of the Current Invention

Any method suitable for detecting protein-protein interactions may be employed for identifying intracellular peptides and proteins that interact with the instant gene products. Among the traditional methods that may be employed are co-immunoprecipitation, crosslinking, and co-purification through gradients or chromatographic columns, of cell lysates (or proteins obtained from cell lysates) and the instant gene product of interest to identify proteins in the cell lysate that interact with the instant gene product of interest. In such assays, the instant gene products may be full length, truncated, modified, part of a fusion protein, or a complex of two or more of the instant gene products. Once isolated, such an intracellular protein can be identified using standard techniques, and/or used again in such an assay to identify other intracellular proteins with which it interacts. For example, at least a portion of the amino acid sequence of an intracellular protein that interacts with one or more of the instant gene products can be ascertained using techniques well-known to those of skill in the art, such as Edman degradation (see, e.g., “Proteins: Structures and Molecular Principles”, supra, pp. 34-49). The amino acid sequence obtained may be used as a guide for the generation of oligonucleotide mixtures that can be used to screen for nucleotide sequences encoding such intracellular proteins, for example, using standard hybridization or PCR techniques (see, e.g., “Current Protocols in Molecular Biology”, supra, and “PCR Protocols: A Guide to Methods and Applications” (Innis et al., eds., Academic Press, Inc., N.Y., 1990)).

Additionally, methods may be employed that result in the simultaneous identification of intracellular proteins, and nucleotide sequences that encode them, that interact with the instant gene products. These methods include, for example, probing expression libraries, in a manner similar to antibody probing of λgt11 libraries, using a labeled form of a gene product of the current invention, or a fusion protein, e.g., a gene product of the invention fused to a marker (e.g., an enzyme, fluor, luminescent protein, or dye) or Ig-Fc domain.

One method that detects protein interactions in vivo, the two-hybrid system, is described in detail for illustration only, and not by way of limitation. One version of this system has been described (Chien et al., Proc. Natl. Acad. Sci. USA 88:9578-9582, 1991) and is commercially available from Clontech. Briefly, utilizing such a system, plasmids are constructed that encode two hybrid proteins: one plasmid consists of nucleotides encoding the DNA-binding domain of a transcription activator protein fused to a nucleotide sequence encoding a gene product of the current invention; and the other plasmid consists of a nucleotide sequence encoding the activation domain of the transcription activator protein fused to a cDNA encoding an unknown protein to be tested for interaction with the gene product of the present invention, which has been recombined into this plasmid as part of a cDNA library. The DNA-binding domain/instant gene product fusion plasmid and the activation domain/cDNA library plasmids are transformed into a strain of the yeast Saccharomyces cerevisiae that contains a reporter gene (e.g., HBS or lacZ) whose regulatory region contains the binding site for the transcription activator protein. Either hybrid protein alone cannot activate transcription of the reporter gene: the DNA-binding domain hybrid cannot because it does not provide transcription activation function; and the activation domain hybrid cannot because it cannot localize to the binding site of the transcription activator protein. Interaction of the two hybrid proteins reconstitutes a functional transcription activator protein, resulting in reporter gene expression that can be detected by an assay for the reporter gene product.

For example, and not by way of limitation, a nucleotide sequence encoding a gene product of the present invention can be cloned into a vector such that it is translationally fused to DNA encoding the DNA-binding domain of the GAL4 protein. A cDNA library from the cell line from which proteins that interact with the instant gene product are to be detected can be made using methods routinely practiced in the art. In this particular example, the cDNA library could be made by inserting the cDNA fragments from the cell line into a vector such that they are translationally fused to the transcription activation domain of the GAL4 protein. This library can be co-transformed along with the plasmid encoding the GAL4 DNA-binding domain/instant gene product fusion into a yeast strain that cannot grow without added histidine, and that contains a HIS3 gene driven by a promoter that contains a GAL4 activation sequence. A cDNA encoded protein, fused to the GAL4 transcriptional activation domain, that interacts with the instant gene product/GAL4 DNA binding domain fusion will reconstitute an active GAL4 protein, and thereby drive expression of the HIS3 gene. Colonies that express HIS3 can be detected by their growth on petri dishes containing semi-solid agar based media lacking histidine. The cDNA can then be purified and sequenced from these colonies, and used to produce and isolate the protein that interacts with the gene product of the current invention using standard techniques.

### 6.6.3 Assays for Compounds that Disrupt the Interaction of the Instant Gene Products and Intracelllar Macromolecules

Macromolecules that interact with the gene products of the current invention are referred to, for purposes of this discussion, as “binding partners”. These binding partners are likely to be involved in catalytic reactions and/or signal transduction pathways, and, thus, in the role of the instant gene products in development and cell differentiation. Compounds that interfere with or disrupt the interaction of such binding partners with the instant gene products may be useful in regulating the activity of the instant gene products, and thus be involved in development and/or cell differentiation disorders.

Certain assay systems used to identify compounds that interfere with or disrupt the interaction between the instant gene products and its binding partner or partners involve preparing a reaction mixture containing the instant gene product of interest and the binding partner under conditions and for a time sufficient to allow them to interact and bind, thus forming a complex. To test a compound for the ability to interfere with complex formation, a reaction mixture is prepared in the presence of the test compound, and a control reaction mixture is prepared in the absence of the test compound, or with a placebo. The test compound may be initially included in the reaction mixture, or added to the reaction mixture after addition of the gene product of interest and its binding partner. The formation of a complex between the instant gene product of interest and the binding partner is then detected. Formation of a complex in the control reaction, but not in the reaction mixture containing the test compound, indicates that the compound interferes with the interaction of the instant gene product of interest and the binding partner. Additionally, complex formation in reaction mixtures containing a test compound and a normal gene product of the current invention can be compared to complex formation in reaction mixtures containing the test compound and a mutant gene product of the current invention, which may be important when it is desirable to identify compounds that disrupt interactions of mutant, but not normal, forms of a gene product of the current invention.

Assays for compounds that interfere with the interaction of a gene product of the current invention and its binding partner(s) can be conducted in a number of ways, including, but not limited to, heterogeneous or homogeneous formats. Heterogeneous assays can involve anchoring either the instant gene product of interest or the binding partner onto a solid phase, and detecting complexes anchored on the solid phase at the end of the reaction, or the entire reaction can be carried out in a liquid phase. In homogeneous assays, the entire reaction is carried out in a liquid phase. Heterogeneous assay formats are detailed above in Section 6.6.1.

In either approach, the order of addition of reactants can be varied to obtain different information about the compounds being tested. For example, test compounds that interfere with the interaction by competition can be identified by conducting the reaction in the presence of the test substance, i.e., adding the test substance to the reaction mixture prior to or simultaneously with the instant gene product of interest and the binding partner, while test compounds that disrupt preformed complexes, e.g., compounds with higher binding constants that displace one of the components from the complex, can be tested by adding the test compound to the reaction mixture after complex formation.

In homogeneous assays, a preformed complex of the instant gene product of interest and the binding partner is prepared in which one of these components is labeled, but the signal generated by the label is quenched due to formation of the complex (see, e.g., U.S. Pat. No. 4,190,496, which utilizes this approach for immunoassays). The addition of a test compound that competes with and displaces one component from the preformed complex will result in the generation of a signal above background. In this way, a test compound that disrupts the interaction between the instant gene product of interest and the binding partner can be identified.

In a particular embodiment, a gene product of the current invention can be prepared for immobilization by fusing it to a glutathione-S-transferase (GST), using a fusion vector such as pGEX-5×-1, such that the binding activity of the gene product of the present invention is maintained in the resulting GST fusion protein. The binding partner can be purified and used to raise a monoclonal antibody, using standard methods as described herein, which can be labeled with a radioactive isotope, for example ¹²⁵I, using standard methods routinely practiced in the art. In a heterogeneous assay, the GST fusion protein can be anchored to glutathione-agarose beads. The binding partner can then be added in the presence or absence of a test compound in a manner that allows interaction and binding to occur. At the end of the reaction, unbound material can be washed away, and the labeled monoclonal antibody added and allowed to bind complexed components. The interaction between the instant gene product of interest and the binding partner can be detected by measuring the amount of radioactivity remaining with the glutathione-agarose beads. Successful inhibition of the interaction by the test compound will result in a decrease in measured radioactivity.

Alternatively, the GST fusion protein and the binding partner can be mixed together in a liquid phase in the absence of the solid glutathione-agarose beads. The test compound can be added either during or after the components are allowed to interact. This mixture can then be added to the glutathione-agarose beads, and unbound material washed away. Once again, the extent of inhibition of complex formation can be detected by adding the labeled antibody, and measuring the amount of radioactivity remaining with the glutathione-agarose beads.

These same techniques can also be employed using peptide fragments corresponding to the binding domains of a gene product of the current invention and/or the binding partner (in cases where the binding partner is a protein), in place of one or both of the full-length proteins. A number of methods routinely practiced in the art can be used to identify and isolate the binding domain(s). These methods include, but are not limited to, mutagenesis of a nucleotide sequence encoding one of the components, and screening for disruption of binding in a co-immunoprecipitation assay. Compensating mutations in the sequence encoding the second component in the complex can then be selected. Sequence analysis of the respective coding sequences will reveal the region of the proteins that contain the mutation(s), and are thus the regions of the proteins involved in interactive binding. Alternatively, one protein can be anchored to a solid surface and allowed to interact with and bind to the binding partner, which has been labeled and treated with a proteolytic enzyme, such as trypsin. After washing, a labeled peptide comprising the binding domain may remain associated with the solid material, which can be isolated and identified by amino acid sequencing. Also, once the nucleotide sequence encoding the binding partner is obtained, short nucleotide segments can be engineered to express peptide fragments of the protein, which can be tested for binding activity, and then purified and/or synthesized.

For example, and not by way of limitation, a peptide or protein of the current invention can be anchored to a solid material by making a GST fusion protein, as described above, and allowing it to bind to glutathione-agarose beads. The binding partner can be labeled with a radioactive isotope, such as ³⁵S, and cleaved with a proteolytic enzyme, such as trypsin. Cleavage products can then be added to the anchored GST fusion protein and allowed to bind. After washing away unbound peptides, labeled bound material, representing the binding domain of the binding partner, can be eluted and purified. The amino acid sequence of the binding domain can be determined using well-known methods, and subsequently produced synthetically, or fused to appropriate facilitative proteins using recombinant DNA technology.

### 6.6.4 Assays for Compounds that Ameliorate Development and/or Cell Differentiation Disorders

Compounds, including, but are not limited to, binding compounds identified via assay techniques such as those described above, can be tested for the ability to ameliorate development and/or cell differentiation disorder symptoms. The invention encompasses cell-based and animal model-based assays for identification of such compounds. Such compounds can be used in a therapeutic method for the treatment of developmental and/or cell differentiation disorders.

Cell-based systems can be used to identify compounds that ameliorate developmental and/or cell differentiation disorder symptoms. Such cell-based systems can include, for example, recombinant or non-recombinant cells, such as cell lines, that express a nucleotide sequence encoding the gene product of interest of the current invention. For example, ES cells, or cell lines derived from ES cells, can be used. In addition, host cells (e.g., fibroblasts, or COS, CHO, or Sf9 cells) genetically engineered to express a functional gene product of the current invention, in addition to factors necessary for the instant gene product to carry out its physiological role, for example signal transduction or catalysis, can be used as an end point in the assay. Such cell-based assay systems can also be used to assay the purity and potency of a natural ligand, or a catalytic subunit or catalytic subunit mutant, including recombinant or synthetic catalytic subunits.

In such cell-based systems, cells may be exposed to a test compound at a concentration and for a time sufficient to ameliorate developmental and/or cell differentiation disorder symptoms in the exposed cells. After exposure, the cells can be examined to determine whether one or more developmental or cell differentiation disorder-like cellular phenotype has been altered to resemble a more normal or wild-type phenotype, or a phenotype more likely to produce a lower incidence or severity of disorder symptoms. Compounds that elicit such an effect are valuable candidates as therapeutics. The cells can also be assayed to measure alterations in the expression of the instant gene product of interest of the current invention, e.g., by assaying cell lysates for the appropriate mRNA transcripts (e.g., by Northern analysis), or alterations in the activity of the gene product of interest of the current invention in the cell. Alternatively, a change in morphology, or alterations in the expression and/or activity of components of pathways or functions that involve the instant gene product of interest, can be assayed in such cells.

In addition, animal-based systems, which may include, for example, mice, may be used to identify compounds capable of ameliorating development and/or cell differentiation disorder symptoms. For example, animals may be exposed to a test compound at a concentration and for a time sufficient to ameliorate developmental and/or cell differentiation disorder symptoms in the exposed animals. After exposure, the animals can be examined to determine whether one or more developmental and/or cell differentiation disorder-like phenotype has been altered to resemble a more normal or wild-type phenotype, a phenotype more likely to produce a lower incidence or severity of disorder symptoms, or reversal of disorder symptoms. Once again, compounds that elicit such an effect are valuable candidates as therapeutics. Such animal models may thus be used as test systems for the identification therapies and interventions that may be effective in treating developmental and/or cell differentiation disorders. Any compound that reverses any aspect of development and/or cell differentiation disorder-like symptoms should be considered a candidate for human developmental and/or cell differentiation disorder therapeutic intervention. Dosages of such compounds may be determined using dose-response curves, as discussed below.

### 6.7 Treatment of Development and Cell Differentiation Disorders

The invention also encompasses methods and compositions for modifying development and/or cell differentiation, and thus treating development and cell differentiation disorders. For example, the level of expression of one or more of the instant gene products can be decreased, and/or the activity of one or more of the instant gene products can be downregulated. Thus, the response of cells, for example ES cells, to factors that activate the physiological responses that enhance the pathological processes leading to developmental and/or cell differentiation disorders may be reduced, and the symptoms ameliorated. Conversely, the response of cells, for example ES cells, to physiological stimuli involving any of the instant gene products and necessary for proper developmental and/or cell differentiation processes may be augmented by increasing the expression and/or activity of one or more of the instant gene products.

### 6.7.1 Inhibition of the Instant Gene Products to Reduce Development and Cell Differentiation Disorder Systems

Any method that neutralizes the catalytic and/or signal transduction activity of the instant gene products, or that inhibits expression of sequences encoding the instant gene products (either transcription or translation), can be used to reduce development and cell differentiation disorder symptoms.

For example, immunotherapy can be designed to reduce the level of endogenous expression of the instant gene products, e.g., using antisense or ribozyme approaches to inhibit or prevent translation of mRNA, triple helix approaches to inhibit transcription, or targeted homologous recombination to inactivate the coding sequence or its endogenous promoter.

Antisense approaches involve oligonucleotides (either DNA or RNA) that are complementary to mRNA specific for the instant gene product of interest. The oligonucleotides will bind to the complementary mRNA transcripts, and prevent translation. Absolute complementarity, although preferred, is not required. A sequence “complementary” to a portion of an RNA, as referred to herein, means a sequence having sufficient complementarity to be able to hybridize with the RNA, forming a stable duplex. In double-stranded antisense nucleic acids, a single strand of the duplex DNA may be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated region (UTR) up to and including the AUG initiation codon, generally are the most efficient in inhibiting translation. Such oligonucleotides should generally include the complement of the AUG start codon. However, sequences complementary to 3′ untranslated sequence have also been shown to be effective in inhibiting translation (see, e.g., Wagner, Nature 372:333-335, 1994). Furthermore, although oligonucleotides complementary to coding regions are usually less efficient in inhibiting translation, these can also be used in accordance with the invention. The antisense nucleic acids should generally be from at least 6 to about 50 nucleotides in length, while in certain embodiments the antisense nucleic acid is at least 10, at least 17, at least 25, or at least 50 nucleotides in length.

In general, in vitro studies are performed to quantitate the ability of the antisense oligonucleotide to inhibit gene expression, utilizing controls that can distinguish between antisense gene inhibition and nonspecific biological effects of the oligonucleotide. Usually these studies compare levels of the target RNA or protein to an internal control RNA or protein, and the results obtained with the antisense oligonucleotide are compared to those obtained with a control oligonucleotide. The control oligonucleotide should be about the same length as the antisense oligonucleotide, and differ in sequence from the antisense oligonucleotide just enough to prevent specific hybridization to the target sequence.

Antisense oligonucleotides can be DNA or RNA, or chimeric mixtures, derivatives, or modified versions thereof, and be single-stranded or double-stranded. The oligonucleotide can be modified at the base moiety, sugar moiety, or phosphate backbone, for example to improve stability of the molecule or hybridization. The oligonucleotide may be conjugated to other groups, such as peptides (e.g., to target host cell receptors in vivo), agents facilitating transport across cell membranes (see, e.g., Letsinger et al., Proc. Natl. Acad. Sci. USA 86:6553-6556, 1989, Lemaitre et al., Proc. Natl. Acad. Sci. USA 84:648-652, 1987, and PCT Application No. WO 88/09810), hybridization-triggered cleavage agents (see, e.g., Krol et al., BioTechniques 6:958-976, 1988), and intercalating agents (see, e.g., Zon, Pharm. Res. 5:539-549, 1988).

The antisense oligonucleotide may comprise at least one modified base moiety, including, but are not limited to, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, N6-adenine, β-D-galactosylqueosine, N6-isopentenyladenine, wybutoxosine, 1-methylguanine, 1-methylinosine, 2,2-dimethylguanine, uracil-5-oxyacetic acid, 2-methyladenine, 2-methylguanine, queosine, β-D-mannosylqueosine, pseudouracil, 3-methylcytosine, uracil-5-oxyacetic acid methylester, 5-methylcytosine, 2-thiouracil, 5-methoxyuracil, 7-methylguanine, 5-methylaminomethyluracil, 5-methoxyaminomethyl-2-thiouracil, 5-methyluracil, inosine, 5′-methoxycarboxymethyluracil, 2-thiocytosine, 4-thiouracil, 5-methyl-2-thiouracil, 2-methylthio-N6-isopentenyladenine, pseudouracil, 3-(3-amino-3-N-2-carboxy propyl)uracil, and 2,6-diaminopurine.

The antisense oligonucleotide may comprise at least one modified sugar moiety, including, but not limited to, hexose, arabinose, 2-fluoroarabinose, and xylulose. The antisense oligonucleotide may comprise at least one modified phosphate backbone, such as phosphorothioate, phosphoramidate, alkyl phosphotriester, phosphorodithioate, phosphoramidothioate, phosphordiamidate, methylphosphonate, formacetal, or analog thereof. Additionally, the antisense oligonucleotide may be an alpha-anomeric oligonucleotide, which forms specific double-stranded hybrids with complementary RNA in which, contrary to the usual alpha-units, the strands run parallel to each other (Gautier et al., Nucl. Acids Res. 15:6625-6641, 1987), 2′-0-methylribonucleotide (Inoue et al., Nucl. Acids Res. 15:6131-6148, 1987), or a chimeric RNA-DNA analogue (Inoue et al., FEBS Lett. 215:327-330, 1987).

Oligonucleotides may be synthesized by standard methods known in the art, e.g., by use of an automated DNA synthesizer (commercially available from Biosearch Technologies, Inc. (Novato, Calif.), Applied Biosystems, etc.). For example, methods for preparing phosphorothioate (Stein et al., Nucl. Acids Res. 16:3209-3221, 1988), or methylphosphonate (using controlled pore glass polymer supports; Sarin et al., Proc. Natl. Acad. Sci. USA 85:7448-7451, 1988) oligonucleotides may be used.

The antisense oligonucleotides should be delivered to cells that express the instant gene products in vivo, for example ES cells. A number of methods have been developed for delivering antisense DNA or RNA to cells, including, but not limited to, direct injection of antisense oligonucleotides into the tissue or cell derivation site (e.g., bone marrow), and systemic administration of antisense oligonucleotides that are modified to target a desired cell (e.g., oligonucleotides linked to peptides or antibodies that specifically bind to receptors or antigens expressed on the target cell surface).

One approach for achieving intracellular concentrations of an antisense oligonucleotide that is sufficient to suppress translation of endogenous mRNAs utilizes a recombinant DNA construct in which the antisense oligonucleotide is placed under the control of a strong pol III or pol II promoter. Such a vector can be introduced in vivo and taken up by a cell that directs the transcription of an antisense RNA. Such a vector can remain episomal, or integrate into a chromosome, as long as it can be transcribed to produce the desired antisense RNA, and can be constructed using standard recombinant DNA techniques. Vectors for use in replication and expression in mammalian cells can be derived from plasmid, viral, cosmid, YAC, or other sources known in the art.

Any promoter, either inducible or constitutive, known to work in mammalian, preferably human, cells can be used to express the sequence encoding the antisense RNA. Such promoters include, but are not limited to, the SV40 early promoter region (Benoist and Chambon, Nature 290:304-310, 1981), the promoter contained in the 3′ long terminal repeat of Rous sarcoma virus (Yamamoto et al., Cell 22:787-797, 1980), the herpes thymidine kinase promoter (Wagner et al., Proc. Natl. Acad. Sci. USA 78:1441-1445, 1981), and the regulatory sequences of the metallothionein gene (Brinster et al., Nature 296:39-42, 1982). The recombinant DNA construct can be introduced directly into the tissue or cell derivation site. Alternatively, viral vectors can be used that selectively infect the desired tissue or cell-type (e.g., viruses that infect cells of hematopoietic lineage), and thus administered by another route (e.g., systemically).

Ribozyme molecules designed to catalytically cleave mRNA transcripts specific for a gene product of the current invention can also be used to prevent translation of the mRNA, or expression of the gene product encoded by the mRNA (see, e.g., PCT Application No. WO 90/11364, and Sarver et al., Science 247:1222-1225, 1990). Ribozymes that cleave mRNA at site specific recognition sequences, as well as hammerhead ribozymes, can be used to destroy the target mRNA. Hammerhead ribozymes cleave mRNA at a location dictated by flanking regions that form complementary base pairs with the target mRNA (see, e.g., Haseloff and Gerlach, Nature 334:585-591, 1988). The sole requirement is that the target mRNA have a 5′-UG-3′ sequence. The ribozyme can be engineered so that the cleavage recognition site is located near the 5′ end of the target mRNA, i.e., to increase efficiency and minimize the intracellular accumulation of non-functional mRNA transcripts.

The ribozymes of the present invention also include RNA endoribonucleases (hereinafter “Cech-type ribozymes”), such as the one that occurs naturally in Tetrahymena thermophila (known as the IVS, or L-19 IVS RNA; see, e.g., Zaug et al., Science 224:574-578, 1984, Zaug and Cech, Science 231:470-475, 1986, Zaug et al., Nature 324:429-433, 1986, Been and Cech, Cell 47:207-216, 1986, and PCT Application No. WO 88/04300). The Cech-type ribozymes have an eight base pair active site that hybridizes to a target RNA sequence, whereupon cleavage of the target RNA takes place. The invention encompasses Cech-type ribozymes that target eight base-pair active site sequences present in mRNA specific for a gene product of interest of the current invention.

As detailed above for antisense oligonucleotides, the ribozymes can comprise modified oligonucleotides (e.g., for improved stability, targeting, etc.), and should be delivered to cells that express the instant gene product of interest in vivo, for example ES cells. A preferred method of delivery involves using a DNA construct “encoding” the ribozyme under the control of a strong constitutive pol III or pol II promoter, as detailed above for antisense oligonucleotides. Because ribozymes, unlike antisense molecules, are catalytic, lower intracellular concentrations are required.

Endogenous expression of a gene of interest can also be reduced by targeting deoxyribonucleotide sequences that are complementary to the regulatory region of the gene (i.e., the promoter and/or enhancers), to form triple helical structures that prevent transcription of the gene of interest in target cells in the body (see, e.g., Helene, Anticancer Drug Des. 6:569-584, 1991, Helene et al., Ann. N.Y. Acad. Sci. 660:27-36, 1992, and Maher, Bioassays 14:807-815, 1992).

Endogenous gene expression can also be reduced by inactivating, or “knocking out”, a nucleotide encoding a gene product of the present invention, or its promoter, using targeted homologous recombination (see, e.g., Smithies et al., Nature 317:230-234, 1985, Thomas and Capecchi, Cell 51:503-512, 1987, and Thompson et al., supra). For example, a sequence encoding a mutant, non-functional gene product of interest of the current invention, or a completely unrelated DNA sequence, flanked by DNA homologous to the endogenous gene encoding the gene product of interest of the current invention (either the coding region or regulatory regions of the gene) can be used, with or without a selectable marker and/or a negative selectable marker, to transfect cells that express the instant gene product of interest in vivo. Insertion of the DNA construct into the endogenous gene, via targeted homologous recombination, results in inactivation of the targeted endogenous gene. While modifications to ES cells can be used to generate animal offspring with an inactive copy of a gene encoding a gene product of interest of the current invention (see, e.g., Thomas and Capecchi, supra, and Thompson et al., supra), this approach can be adapted for use in humans using appropriate viral vectors that are directly administered, or targeted to the desired site in vivo.

Additionally, the activity of an instant gene product of interest can be reduced using a “dominant negative” approach. A dominant negative approach takes advantage of the interaction of an instant gene product of interest with other peptides or proteins to form a complex that is a prerequisite for the instant gene product of interest to exert its physiological activity. To this end, constructs that encode a defective form of the instant gene product of interest can be used in gene therapy approaches to diminish the activity of the instant gene product of interest in appropriate target cells. Alternatively, targeted homologous recombination can be used to introduce a sequence encoding a defective form of the instant gene product of interest into the endogenous gene encoding the instant gene product of interest in one or more desired tissue(s). The engineered cells will express non-functional copies of the instant gene product of interest, thereby downregulating its activity in vivo. Such engineered cells can demonstrate a diminished response to physiological stimuli of the activity of the affected instant gene product of interest, resulting in reduction of a developmental and/or cell differentiation disorder phenotype.

### 6.7.2 Increasing the Expression or Activity of the Instant Gene Products to Promote Development or Cell Differentiation

With respect to an increase in the level of normal gene expression and/or gene product activity specific for any of the gene products of interest of the current invention, the respective nucleic acid sequences can be utilized for the treatment of development and cell differentiation disorders. Where the cause of the development or cell differentiation dysfunction is a defective gene product of the current invention, treatment can be administered, for example, in the form of gene delivery or gene therapy. Specifically, one or more copies of a normal nucleic acid sequence, or fragment thereof, that directs production of a gene product exhibiting normal function of the appropriate gene product of the current invention, may be inserted into the appropriate cells within a patient or animal subject, for example using suitable vectors.

Recombinant retroviruses have been widely used in gene transfer or gene delivery experiments, and even human clinical trials (see, e.g., Mulligan, in “Experimental Manipulation of Gene Expression”, Chapter 8, pp. 155-173 (Inouye, ed., Academic Press, N.Y., 1983), and Coffin, in “RNA Tumor Viruses”, Vol. 2, pp. 36-38 (Weiss et al., eds., Cold Spring Harbor Press, Cold Spring Harbor, N.Y., 1985)). Other eucaryotic viruses that have been used as vectors to transduce mammalian cells include, but are not limited to, adenovirus, papilloma virus, herpes virus, adeno-associated virus, vaccinia virus, and rabies virus (see, e.g., “Molecular Cloning: A Laboratory Manual”, Vol. 3, Chapter 16, supra).

Alternatively, cationic or other lipids may be employed to deliver polynucleotides encoding the instant gene product of interest to patients. Additionally, “naked” DNA comprising one or more polynucleotides encoding the instant gene products of interest, optionally operably linked to one or more of a promoter, an enhancer, a ribosome entry or ribosome binding site, and/or an in-frame translation initiation codon, can be used for delivery to a patient. Such “naked” DNA vaccines can be delivered in vivo alone, or in conjunction with excipients, microcarrier spheres, nanoparticles, or other supporting or dosaging compounds or molecules.

The described gene replacement/delivery therapies should be capable of delivering polynucleotides to cell-types within patients that express the gene product of interest of the current invention. Targeted homologous recombination, as described above, can also be utilized to correct a defective endogenous gene in the appropriate cell-type. In animals, targeted homologous recombination can be used to correct a defect in ES cells, to produce progeny with a corrected trait.

Finally, compounds identified in the assays described herein that stimulate, enhance, or modify the activity of the gene product of interest of the current invention can be used to correct or ameliorate development and cell differentiation disorders. The formulation and mode of administration will depend upon the physico-chemical properties of the compound.

### 6.8 Pharmaceutical Compositions and Methods of Administration

Compositions that affect expression or activity of the gene products of the current invention, or the interaction of the gene products of the present invention with any of their binding partners, or that comprise one or more nucleotide sequences encoding a gene product of the present invention (i.e., sequences used in antisense, gene therapy, dsRNA, or ribozyme applications), can be administered to a patient at therapeutically effective doses to treat or ameliorate development and/or cell differentiation disorders. A therapeutically effective dose refers to an amount of the compound or composition that results in any amelioration or retardation of development, cell differentiation and/or proliferation disorders or disease symptoms.

### 6.8.1 Effective Dose

Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD₅₀/ED₅₀. Compositions exhibiting large therapeutic indices are preferred. While compositions that exhibit toxic side effects may be used, care should be taken to design a delivery system that targets such compositions to the site of affected tissue, in order to minimize potential damage to uninfected cells and, thereby, reduce side effects.

The data obtained from the cell culture assays and animal studies can be used in formulating a dosage range for use in humans. The dosage of such compositions lies preferably within a range of circulating concentrations that include the ED₅₀ with little or no toxicity. The dosage may vary within this range depending upon the dosage form employed and the route of administration utilized. For any compositions used in such treatment methods, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human efficacy studies begin, Phase I clinical studies in normal subjects help establish safe doses.

The bioactive agent may be complexed with a variety of compounds or structures known in the art that, for instance, enhance the stability of the bioactive agent, or otherwise enhance its pharmacological properties (e.g., increase in vivo half-life, reduce toxicity, etc.). The therapeutic agents can be administered by any number of methods known to the skilled artisan, including, but not limited to: subcutaneous (sub-q), intravenous (I.V.), intraperitoneal (I.P.), intramuscular (I.M.), or intrathecal injection; inhalation; or topically (transderm, ointments, creams, salves, eye drops, etc.).

### 6.8.2 Formulations and Use

Pharmaceutical compositions for use in the present invention may be formulated in a conventional manner using one or more physiologically acceptable carriers or excipients. The compositions, or their physiologically acceptable salts or solvates, may be formulated for administration by any route, such as inhalation or insufflation (either through the mouth or nose), oral, buccal, parenteral, or rectal administration.

For administration by inhalation, the compositions are conveniently delivered in the form of an aerosol spray from pressurized packs or a nebulizer, with the use of a suitable propellant, e.g., dichlorodifluoromethane, carbon dioxide, trichlorofluoromethane, dichlorotetrafluoroethane, or other suitable gas. In the case of a pressurized aerosol the dosage unit may be determined by providing a valve to deliver a metered amount. Capsules and cartridges for use in an inhaler or insufflator may be formulated with a powder mix of the compound and a suitable powder base such as lactose or starch.

For oral administration, the compositions can take the form of tablets or capsules with pharmaceutically acceptable excipients, such as binding agents (e.g., pregelatinized maize starch, polyvinylpyrrolidone, hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of solutions, syrups, or suspensions, or they may be presented as a dry product for constitution with water or other suitable vehicle before use. Such liquid preparations may comprise pharmaceutically acceptable additives, such as suspending agents (e.g., sorbitol syrup, cellulose derivatives or hydrogenated edible fats); emulsifying agents (e.g., acacia or lecithin); non-aqueous vehicles (e.g., almond oil, oily esters, ethyl alcohol, or fractionated vegetable oils); or preservatives (e.g., methyl or propyl-p-hydroxybenzoates or sorbic acid). The preparations may also contain buffer salts, flavoring, coloring and sweetening agents as appropriate. Preparations for oral administration may be formulated to give controlled release of the active compound.

For buccal administration the compositions may take the form of tablets or lozenges formulated in conventional manner.

For parenteral administration, the compositions may be formulated for injection, e.g., by bolus injection or continuous infusion. Such formulations may be presented in unit dosage form, e.g., in ampules or multi-dose containers, with an added preservative. The compositions may take such forms as suspensions, solutions, or emulsions in oily or aqueous vehicles, and may contain formulatory agents such as suspending, stabilizing and/or dispersing agents. The active ingredient may also be in powder form for constitution with a suitable vehicle, like sterile pyrogen-free water, before use.

For rectal administration, compositions may be formulated as suppositories or retention enemas, e.g., containing suppository bases such as cocoa butter or other glycerides.

The pharmaceutical compositions may also be formulated as a depot preparation. Such long acting formulations may be administered by implantation (e.g., sub-q or I.M.), or by intramuscular injection. The compositions may be formulated with suitable polymeric or hydrophobic materials (for example as an emulsion in an acceptable oil),ion exchange resins, or as sparingly soluble derivatives, for example, as a sparingly soluble salt. The compositions may be presented in a pack or dispenser device that contains one or more unit dosage forms containing the active ingredient. The pack may comprise metal or plastic foil, like a blister pack. The pack or dispenser device may be accompanied by instructions for administration.

### 6.9. Analysis of the Human Genome Using GTS Sequences

The Human Genome Project and privately financed ventures have provided the world with a copy of the sequence of each human chromosome. However, the overwhelming majority of this genomic sequence information does not encode proteins, and the portion that does encode proteins is generally dispersed as exons amidst large regions of intervening intronic sequence. Thus, to effectively make genomic sequence information useful, identification of the coding regions, splice junctions, and chromosomal location will generally be necessary.

Within any cell in the human body there are estimated to be 10,000-20,000 different genes that are actually expressed among a estimated 50,000-125,000 total genes. Within this group, there are genes that are abundantly expressed, such as “house-keeping” genes, those that are moderately expressed, and those that are rarely expressed. Rarely expressed genes are often those that attract the most scientific interest in relation to disease, development, and cell cycle processes. The majority of key proteins in these processes, many of which encode enzymes, are either rarely expressed, or expressed in a tightly controlled fashion. ESTs have been used for identifying coding regions in the human genome, but because of the techniques used for identifying ESTs, the majority of genes that have been so identified are those that are abundantly expressed in the cell. In addition, there has been a huge overlap in identification of those genes at the higher end of the expression curve. Thus, about 30-40% of human genes are still undiscovered, and identification of rarely expressed sequences is a major scientific goal.

The gene trap technology described herein allows for the identification of such rarely expressed sequences. Because the this technology does not rely on endogenous promoters, there is an essentially equal chance of isolating a sequence that is rarely expressed in the cell as a sequence that is highly expressed in the cell. Accordingly, a key advantage of gene trapping is that it allows for the identification and isolation of rarely expressed sequences in a fraction of the time necessary to isolate such sequences previously. For example, of the first 20,000 human genes identified by gene trapping, approximately 10,000 were not present in published gene sequence databases. Therefore, SEQ ID NOS:10-5,504 comprise a significant number of rarely expressed sequences, and allow the study of key elements of cellular processes, as well as the best targets for therapeutic intervention.

To identify a sequence as “rarely expressed”, expression patterns in a variety of tissues, cell lines, and cancer cells can be determined using techniques known to one of skill in the art, including Northern blot technology, Quantitative PCR, and others. Obtaining a weak or nonexistent signal in the majority of cases suggests the sequence is “rarely expressed”. A GTS corresponding to such a sequence would be particularly useful in gene chip or comparable hybridization technology.

The GTSs described herein also do not display the 3′ bias typical of EST sequences obtained by conventional methods, and are thus more likely to provide information from the coding region of genes as opposed to 3′ (or 5′) untranslated regions.

When the GTSs of SEQ ID NOS:10-5,504 are overlaid onto the map of the human genome, a variety of information can be obtained, including, for example, coding regions (exons), splice junctions, and chromosomal locations. To analyze the described GTSs, or overlay them on human genomic sequence, a computer-based system including a search program for accessing a human genome database can be used. The sequence from any of SEQ ID NOS:10-5,504 is compared and aligned to the human genome, allowing for gaps. Where homologous genomic sequence is found, a given GTS typically identifies several dispersed regions of homology, and the intervening gaps will generally constitute introns. Thus, the GTSs can be used to identify the specific locations of exon splice junctions, which can be particularly important in the study of disease and cancer, since splice junctions are often hot spots for erroneous events leading to these disease states.

The GTSs can be used to identify the chromosomal location of a coding region (or an experimentally determined location confirmed), by layering or aligning the GTSs with human genomic sequence, as discussed above, or a specific location on a chromosome can be “searched” for the correct sequence. The chromosomal location is an important piece of information when looking for those regions of the human genome involved in genetic diseases, cancer, and predispositions to various disease states. Chromosomal translocations have been found to be associated with a number of disease states, and often it is possible to locate a gross chromosome position for a disease-associated gene. Thus, this information will help to pinpoint the exact location of a potentially disease-associated gene.

### 6.10 Computer Related Embodiments

The GTSs of SEQ ID NOS:10-5,504, fragments thereof, or nucleotide sequences at least 99% identical thereto, can be “provided” in a variety of media to facilitate use. As used herein, “provided” refers to a manufacture, other than an isolated nucleic acid molecule, that contains a nucleotide sequence of the present invention, as set forth herein. Such a manufacture provides the human genome, or subset thereof (e.g., a human ORF), in a form that allows a skilled artisan to examine the manufacture using means not directly applicable to the human genome as it exists in nature or purified form.

In one embodiment, a nucleotide sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The choice of the medium will generally be based on the means chosen to access the stored information. As used herein, “recorded” refers to any process for storing information on computer readable media. The skilled artisan can generate a manufacture comprising computer readable media recorded thereon a nucleotide sequence of the present invention, using well-known materials and techniques.

A variety of data processing programs and formats can be used to store the instant nucleotide sequence information on computer readable media. The information can be represented in a word processing text file, formatted in commercially-available software, such as WordPerfect or MicroSoft Word, represented in the form of an ASCII file, stored in a database application, such as DB2, Sybase, Oracle, or the like. The skilled artisan can readily adapt any number of data processor structuring formats (e.g., text file or database) to obtain computer readable media having recorded thereon the nucleotide sequence information of the present invention.

By providing the nucleotide sequence of the present invention in computer readable form, a skilled artisan can routinely access the sequence information for a variety of purposes. Computer software is publicly available that allows a skilled artisan to access sequence information provided in a computer readable medium. Software that implements the BLAST (Altschul et al., J. Mol. Biol. 215:403-410, 1990) or BLAZE (Brutlag et al., Comp. Chem. 17:203-207, 1993) search algorithm on a Sybase system can be used to identify ORFs in the Homo sapiens genome that contain homology to ORFs or proteins from other organisms. Such ORFs can be useful in producing commercially important metabolites or proteins, such as enzymes used in fermentation reactions.

The present invention also provides systems, particularly computer-based systems, that contain the sequence information described herein. Such systems are designed to identify commercially important fragments of the Homo sapiens genome. As used herein, a “computer-based system” refers to the hardware means, software means, and data storage means used to analyze the instant nucleotide sequence information. Minimum hardware means of such a computer-based system comprises a central process unit (CPU), input means, output means, and data storage means. As used herein, “data storage means” refers to memory that can store the instant nucleotide sequence information, or a memory access means that can access manufactures having recorded thereon the instant nucleotide sequence information. The skilled artisan readily appreciates that any one of the currently available computer-based systems are suitable for use in the present invention.

The instant computer-based systems also comprise the necessary hardware means and software means for supporting and implementing a search means. As used herein, “search means” refers to one or more programs that are implemented on the computer-based system to compare a target sequence with the sequence information stored within the data storage means. Search means are used to identify fragments or regions of the Homo sapiens genome that match a particular target sequence. A variety of commercially available software for conducting search means can be used in the computer-based systems of the present invention, including, but not limited to, MacPattern (EMBL), BLASTN and BLASTX (NCBIA). The skilled artisan can recognize that any available algorithms or implementing software packages for conducting homology searches can be adapted for use in the present computer-based systems.

As used herein, a “target sequence” can be any nucleic or amino acid sequence of six or more nucleotides or two or more amino acids. A skilled artisan can readily recognize that the longer a target sequence is, the less likely a target sequence will be present as a random occurrence in the database. A preferred length of a target sequence is about 10 to 100 amino acids or about 30 to 300 nucleotide residues. However, it is well-known that searches for commercially important fragments of the Homo sapiens genome, such as sequences involved in gene expression and protein processing, may be of shorter length.

A variety of structural formats for the input and output means can be used in the computer-based systems of the present invention. A preferred format for an output means ranks fragments of the human genome possessing varying degrees of homology to the target sequence. Such presentation provides a skilled artisan with a ranking of sequences that contain various amounts of the target sequence, and identifies the degree of homology contained in the identified fragment.

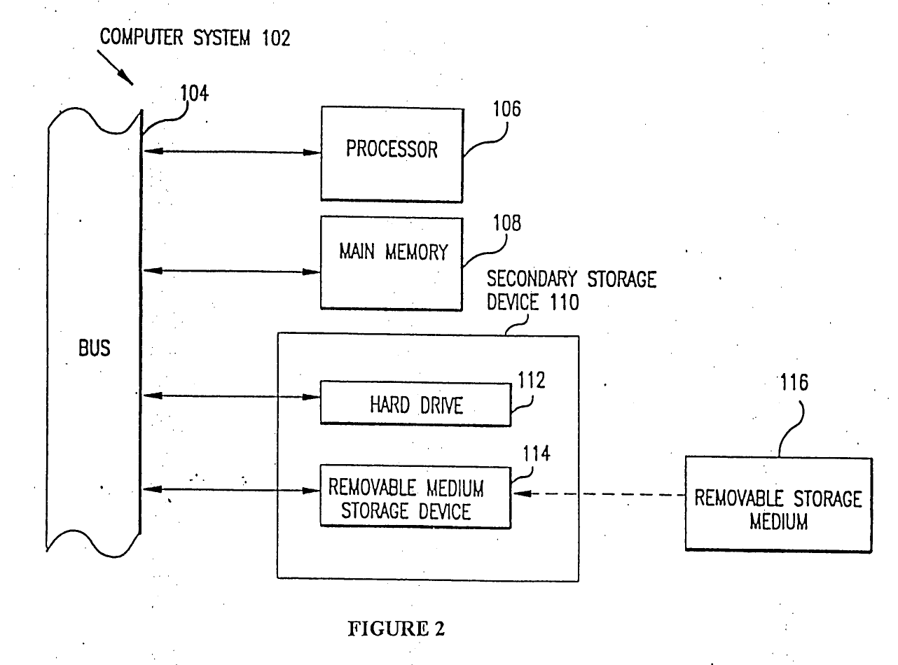

One embodiment of a computer-based system is provided in FIG. 2. FIG. 2 provides a block diagram of a computer system 102 that can be used to implement the present invention. The computer system 102 includes a processor 106 connected to a bus 104. Also connected to the bus 104 are a main memory 108 (preferably implemented as random access memory, RAM) and a variety of secondary storage devices 110, such as a hard drive 112 and a removable medium storage device 114. The removable medium storage device 114 may represent, for example, a floppy disk, CD-ROM, or magnetic tape drive. A removable storage medium 116 (such as a floppy or compact disk or magnetic tape) containing control logic and/or data recorded therein may be inserted into the removable medium storage device 114. The computer system 102 includes appropriate software for reading the control logic and/or the data from the removable medium storage device 114 once inserted in storage device 114. Similar systems are described in U.S. Provisional Application Ser. Nos. 60/044,031, 60/046,655, and 60/066,009.

A nucleotide sequence of the present invention may be stored in a well-known manner in the main memory 108, any of the secondary storage devices 110, and/or a removable storage medium 116. Software for accessing and processing the genomic sequence (such as search tools, comparing tools, etc.) reside in main memory 108 during execution.

The examples below are provided to illustrate the subject invention. These examples are provided by way of illustration only, and are not included for the purpose of limiting the invention in any way whatsoever.

### 7.0 EXAMPLES

### 7.1 Construction of Trapped cDNA Libraries

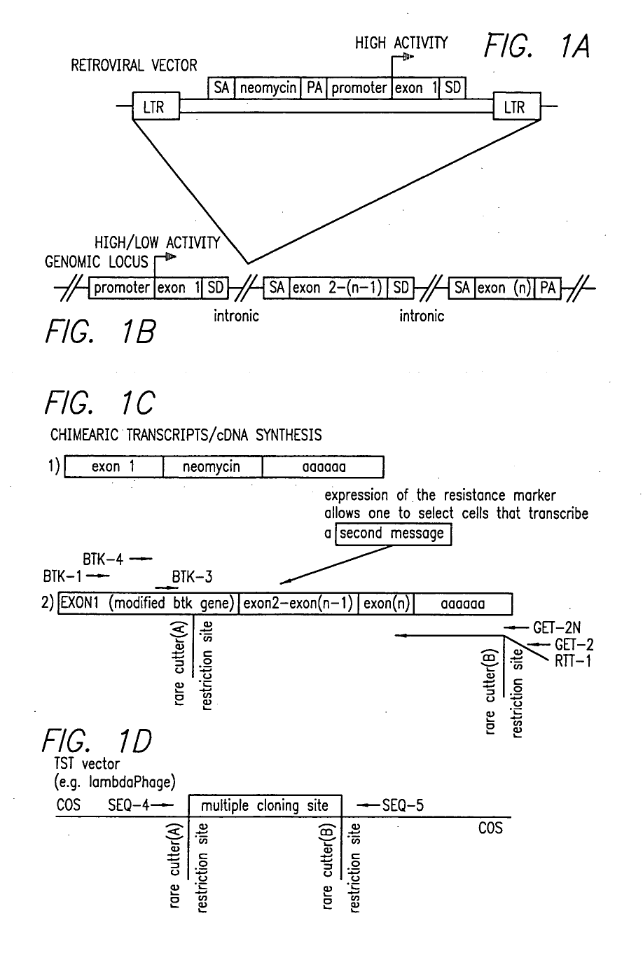

The GTSs of SEQ ID NOS:10-5,504 were generated using normalized cDNA libraries produced as described in U.S. Pat. No. 6,218,123. FIG. 1A is a representative illustration of the retroviral vector used to produce the GTSs. In brief, pools of modified human PA-1 teratocarcinoma cells (PA-2; PA-1 that has been transfected to express the murine ecotropic retrovirus receptor) were typically infected at a multiplicity of infection (MOI) between about 0.01 and about 0.1 (although much higher MOIs such as 1 to more than 10 could have been used). FIG. 1B a schematic of how the target cell genomic locus is presumably mutated by integration of the retroviral construct into intronic sequences of the cellular gene. The integrated retrovirus generates two chimeric transcripts. As illustrated in FIG. 1C, the first chimeric transcript is a fusion between the coding region of the resistance marker (neo in the present case) carried within the transgenic construct and the upstream exon(s) from the cellular gene. A mature transcript is generated when the indicated splice donor (SD) and splice acceptor (SA) sites are spliced. Translation of this fusion transcript produces the protein encoded by the resistance marker, and allows for selection of gene trapped target cells, although selection is not required to produce the described polynucleotides (see below).

The second chimeric transcript is shown in FIG. 1C. This transcript is a fusion of the first exon of the transgenic construct (EXON1; the first exon of the murine btk gene was used as the sequence acquisition component for the described GTSs) with downstream exons from the cellular genome. Unlike the transcript encoding the selectable marker exon, the transcript encoding EXON1 is transcribed under the control of a vector encoded, and hence exogenously added, promoter (such as the PGK promoter), and the corresponding mRNA is generated by splicing between the indicated SD and SA sites. The region encoding the sequence acquisition exon (EXON1) has also been engineered to incorporate a unique sequence that permits the selective enrichment of the fusion transcript using molecular biological methods such as, for example, the polymerase chain reaction (PCR). These sequences are unique primer binding sites for EXON1-specific PCR amplification of the transcript, and can additionally incorporate one or several rare-cutter endonuclease restriction sites to allow site-specific cloning. These features allow for efficient and preferential cloning of transgene-expressed fusion transcripts from pools of target cells, relative to background cellularly-encoded transcripts.

Based on the unique sequence present in EXON1, which is indicated as a rare-cutter (A) restriction site in FIG. 1B, selective cloning of the fusion transcript is achieved as shown in FIG. 1D. cDNA was generated by reverse transcribing isolated RNA from pools of cells that have undergone independent gene trap events using, for example, a primer that consists of a homopolymeric stretch of deoxythymidine residues that bind to the polyadenylated end of the mRNA at the 3′ end, a sequence that serves as a binding site for a second and third primer at the 5′ end, and the sequence of a second rare-cutter (B) restriction site in the center. Depending on the size of the pool and the transcriptional levels of the fusion transcript, second strand synthesis was carried out using either Klenow polymerase, or by PCR. The reaction products are digested with two corresponding rare-cutter restriction endonucleases (e.g., recognize A and B). PCR conditions can be modified to enhance the size of the PCR products, using procedures such as those described in, inter alia, U.S. Pat. No. 5,556,772, and the 1997/98 PanVera New Technologies for Biomedical Research Catalog (Madison, Wis.). Prior to cloning, PCR cDNA fragments can be size selected using conventional methods, such as chromatography or gel electrophoresis. Alternatively, or in addition, to this size selection, PCR templates can first be size selected into separate pools.

After digestion and size selection, the cleaved cDNAs are directionally cloned into phage vectors (FIG. 1D), although other cloning vectors/vehicles could have been used. Such vectors are generically referred to as gene trapped sequence vectors, or “GTS vectors” in FIG. 1D, and generally comprise a multiple cloning site with restriction sites corresponding to those incorporated into the amplified cDNAs (e.g., SfiI, which allows for directional cloning of the cDNAs). After cloning, the resulting phage are handled as a conventional cDNA library using standard procedures. Individual colonies and/or plaques were picked and used to generate PCR-derived templates for DNA sequencing reactions.

A more detailed description of the above follows. The btk gene trap vector/virus containing supernatant from GP+E or AM12 packaging cells was added to approximately 50,000 human PA-2 cells (at an input ratio between about 0.1 and about 0.1 virus/target cell) for about 16 to about 24 hours. The cells were subsequently selected with G418 at active concentration of about 400 μg/ml for about 10 days. Between about 600 and about 3,000 G418 resistant colonies were subsequently pooled, and subjected to RNA isolation, reverse transcription, PCR, restriction digestion, size selection, and subcloning into lambda phage vectors. Individual phage plaques were directly amplified, purified, and sequenced to obtain some of the GTSs.

When selection was not used, about 1×10⁶ cells (PA-2, HeLa, HepG2, or Jurkat) per 100 mm dish were plated and infected with AM12 packaged btk retrovirus at an MOI of about 0.01. After a 16 hour incubation, the cells were washed in PBS and grown in culture media for four days. RNA from each plate was extracted, reverse transcribed, and the resulting cDNA was subject to two rounds of PCR, each for 25 cycles. The resulting PCR products were digested with SfiI and separated by gel electrophoresis. Six size fractions were recovered (between about 300 and about 4,000 bp), which were each ligated into lambdaGT10Sfi arms, in vitro packaged, and plated for lysis. Individual plaques were picked from the plates, subject to an additional round of PCR, and then sequenced to obtain some of the GTSs, as detailed below.

Total cell RNA was isolated using RNAzol (ISO-TEX Diagnostics, Friendswood, Tex.), using the protocol provided. RT premix, containing 2× First Strand buffer (100 mM Tris-HCl, pH 8.3, 150 mM KCl, 6 mM MgCl₂and 2 mM dNTPs), RNAGuard (1.5 units/rxn; Amersham Pharmacia, Piscataway, N.J.), 20 mM DTT, primer (3 pmol/rxn; either RTT-1A; 5′-TGGCTAGGCCCCAGGATAGGCCTC GCTGGCCTTTTTTTTT-3′ (SEQ ID NO:1), or RTT-1B; 5′-TGGCTAGGCCCCA GGATAGGCCTCGCTGGCCTTTTTTTTTTTTTTTTT-3′ (SEQ ID NO:9), Genosys Biotechnologies, Inc., The Woodlands, Tex.), and Superscript II RT (200 units/rxn; Invitrogen Corporation, Carlsbad, Calif.) was added to the RNA, and the RT reaction was carried out in a thermal cycler (37° C. for 5 minutes, 42° C. for 30 minutes, and 55° C. for 10 minutes).

The cDNA was amplified using two distinct, nested, stages of PCR. The PCR premix contained 1.1× MGBII buffer (74 mM Tris, pH 8.8, 18.3 mM (NH₄)₂SO₄, 7.4 mM MgCl₂, 0.011% gelatin, and 5.5 mM β-mercaptoethanol), 11.1% DMSO (Sigma, St. Louis, Mo.), 1.67 mM DNTPS, Taq DNA polymerase (5 units/rxn), water and primers. The first round of PCR used the primers BTK-1 (5′-GCCATGGCTCCGGTAGGTCCAGAG-3′, SEQ ID NO:2) and GET-2 (5′-TG GCTAGGCCCCAGGATAG-3′, SEQ ID NO:3), at about 7 pmol/rxn. The PCR premix with the first round primers was added to an aliquot of cDNA, and run in a thermal cycler for 20 cycles (94° C. for 45 seconds, 56° C. for 60 seconds, and 72° C. for 2-4 minutes). An aliquot of this reaction was added to the PCR premix with the second round primers BTK-4 (5′-GTCCAGAGATGGCCA TAGC-3′, SEQ ID NO:4) and GET-2N (5′-CCAGGATAGGCCTCGCTG-3′, SEQ ID NO:5), at about 20 pmol/rxn, and run in a thermal cycler 20 cycles (94° C. for 45 seconds, 56° C. for 60 seconds, and 72° C. for 2-4 minutes).

The products from the second round of PCR were extracted using phenol/chloroform and chloroform, and then isopropanol precipitated in the presence of glycogen/sodium acetate. After centrifugation, the nucleic acid pellets were washed with 70 percent ethanol, and resuspended in TE buffer, pH 8.0. After digestion with SfiI at 55° C., the digested products were loaded onto 0.8% agarose gels and size-selected using DEAE membranes (“Molecular Cloning: A Laboratory Manual”, supra). Generally, six size fractions (less than 700 bp, 700-900 bp, 900-1,300 bp, 1,300-1,600 bp, 1,600-2,000 bp, and greater than 2,000 bp) were separately ligated into GTS vector arms that were engineered to contain the corresponding SfiI “A” and “B” specific overhangs (i.e., TAG and GCG, respectively). The ligation products were packaged using commercially available lambda packaging extracts (Promega Corporation, Madison, Wis.), and plated using E. coli strain C600 (“Molecular Cloning: A Laboratory Manual”, supra). Individual plaques were directly picked into 40 microliters of PCR buffer and subjected to 35 cycles of PCR (94° C. for 45 seconds, 56° C. for 60 seconds, and 72° C. for 1-3 minutes, depending on the size fraction) using 12 pmol of primers SEQ-4 (5′-TACAGTTTTTCTTGTGAAGATTG-3′, SEQ ID NO:6) and SEQ-5 (5′-GGGTAGTCCCCACCTTTTG-3′, SEQ ID NO:7) per PCR reaction. The cloned 3′ RACE products were purified using an S300 column equilibrated in STE, essentially as previously described (Nehls and Boehm, Trends Genet. 9:336-337, 1993), and the products recovered by centrifugation at 1,200×g for 5 minutes. This step removes unincorporated nucleotides, oligonucleotides, and primer-dimers. The PCR products were subsequently applied to a 0.25 ml bed of Sephadex® G-50 (DNA Grade, Pharmacia Biotech AB, Uppsala, Sweden) that was equilibrated in MilliQ H₂O, and recovered by centrifugation as described above. Purified PCR products were quantified by fluorescence using PicoGreen (Molecular Probes, Inc., Eugene, Oreg.) per the manufacturer's instructions.

Dye terminator cycle sequencing reactions with AmpliTaq® FS DNA polymerase (Applied Biosystems) were carried out using BTK-3 primer (5′-TCCAAGTCCTGGCATCTCAC-3′, SEQ ID NO:8) at 7 pmol and approximately 30-120 ng of 3′ template. Unincorporated dye terminators were removed from the completed sequencing reactions using G-50 columns as described above. The reactions were dried under vacuum, resuspended in loading buffer, and electrophoresed through a 6% Long Ranger acrylamide gel (FMC BioProducts, Rockland, Me.) on an ABI Prism® 377 with XL upgrade (Applied Biosystems) per the manufacturer's instructions. The sequences of the amplicons, or GTSs, are described in SEQ ID NOS:10-5,504.

All publications and patents mentioned in the above specification are herein incorporated by reference in their entirety. In the event that one or more of the incorporated literature and similar materials defines a term in a manner that contradicts the definition of that term in this application, this application controls. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention that are obvious to those skilled in the field of molecular biology or related fields are intended to be within the scope of the following claims.

What is claimed is:

1. An isolated polynucleotide comprising at least 25 contiguous nucleotides of any of SEQ ID NOS:10-5,504, or the complement thereof.

2. The isolated polynucleotide of claim 1, comprising at least 60 contiguous nucleotides of any of SEQ ID NOS:10-5,504, or the complement thereof.

3. The isolated polynucleotide of claim 2, comprising the nucleotide sequence of any of SEQ ID NOS:10-5,504, or the complement thereof.

4. A combination comprising a plurality of cDNAs, wherein said plurality of cDNAs comprises any 15 of SEQ ID NOS:10-5,504, or the complement thereof.

5. The combination of claim 4, wherein said plurality of cDNAs comprises any 25 of SEQ ID NOS:10-5,504, or the complement thereof.

6. The combination of claim 5, wherein said plurality of cDNAs comprises any 50 of SEQ ID NOS:10-5,504, or the complement thereof.

7. The combination of claim 6, wherein said plurality of cDNAs consists of SEQ ID NOS:10-5,504, or the complements thereof.

8. The combination of claim 4, wherein said plurality of cDNAs are immobilized on a substrate.

9. A computer-based system for identifying an exon sequence of the human genome, comprising:
a) a data storage means comprising the nucleotide sequence of any of SEQ ID NOS:10-5,504, or the complement thereof; and
b) search means for comparing a target genomic nucleotide sequence of the human genome to the nucleotide sequence of said data storage means to identify a homologous sequence, wherein the presence of a homologous sequence identifies an exon sequence of the human genome.
